SEQUENCE LISTING (8 sequences)

SEQ ID NO: 1 - protein, 10 aa, Lama glama
GRTFSNYNMG

SEQ ID NO: 2 - protein, 17 aa, Lama glama
AIDWSGGSPYYAASVRG

SEQ ID NO: 3 - protein, 12 aa, Lama glama
PLSYGSTWLADY

SEQ ID NO: 4 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 5 - protein, 297 aa, Macaca fascicularis
MTTPRNSVNGTFPAEPMKGPIAMQPGPKPLLRRMSSLVGPTQSFFMRESKALGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRVHTPYINIYNCEPANPSEKNSPSTQYCYSIQSLFLGILSVMLIFAFFQELVIAGIVENEWRRTCSRPKSSVVLLSAEEKKEQVIEIKEEVVGLTETSSQPKNEEDIEIIPIQEEEEEETETNFPEPPQDQESSPIENDSSP

SEQ ID NO: 6 - protein, 121 aa, Lama glama
QVQLQESGGGLVQAGGSLRLSCAASGRTFSNYNMGWFRQAPGKEREFVAAIDWSGGSPYYAASVRGRFTISRDNAENTVYLQMNSLKPEDTAVYYCAAPLSYGSTWLADYWGQGTQVTVSS

SEQ ID NO: 7 - DNA, 40 bp, Artificial Sequence
  Primer
gataagatctcaggcggatccacaacacccagaaattcag

SEQ ID NO: 8 - DNA, 42 bp, Artificial Sequence
  Primer
ggttttttctctagatcaaggagagctgtcattttctattgg

(19) United States
(12) Patent Application Publication
(10) Pub. No.: US 2019/0077871 A1
(43) Pub. Date: Mar. 14, 2019

(54) CD20 BINDING AGENTS AND USES THEREOF

(71) Applicants: VIB VZM, Gent (BE); UNIVERSITY GENT, Gent (BE); VRIJE UNIVERSITEIT BRUSSEL, Brussel (BE)

(72) Inventors: Jan Tavernier, Balegem (BE); José Van Der Heyden, Munte (BE); Nick Devoogdt, Eppegem (BE); Matthias D'Huyvetter, Antwerpen (BE); Ahmet Krasniqi, Dilbeek (BE)

(21) Appl. No.: 16/082,701

(22) PCT Filed: Mar. 6, 2017
(86) PCT No.: PCT/EP2017/055200

§ 371 Date: Sep. 6, 2018

(30) Foreign Application Priority Data
Mar. 7, 2016 (EP) 16159024.5

(51) Int. Cl. C07K 16/28; A61P 35/00; G01N 33/574; A61K 51/10

(52) U.S. Cl. CPC C07K 16/2887; A61P 35/00; A61K 2039/507; A61K 51/1027; A61K 51/1096; G01N 33/574

(57) ABSTRACT

The present invention relates to radiolabeled binding agents (e.g. antibodies, such as, without limitation, single-domain antibodies) which bind CD20 and their use as diagnostic, prognostic, predictive and therapeutic agents.

### FIELD OF THE INVENTION

The present invention relates to radiolabeled binding agents (e.g. antibodies, such as, without limitation, single-domain antibodies) which bind CD20 and their use as diagnostic, prognostic, predictive and therapeutic agents.

### BACKGROUND

In oncology there is a growing interest in targeted radionuclide therapy (TRNT) that selectively delivers radioactivity and kills malignant cells, while minimizing the harm to healthy cells (Ersahin et al., 2011). Due to the widespread availability of therapeutic radionuclides, this therapy strategy is gaining more attention (Tomblyn et al., 2012). The integration of diagnostic testing (molecular imaging) for the presence of a molecular target is of interest to predict successful TRNT. This so-called theranostic approach aims to improve personalized treatment based on the molecular characteristics of cancer cells. Moreover, this strategy offers new insights in predicting the dose needed to treat and provides appropriate tools to monitor therapy responses.

Radioimmunotherapy (RIT) is a TRNT strategy that employs radiolabeled monoclonal antibodies (mAbs) that interact with tumor-associated proteins that are expressed on the cancer cell surface and thus readily accessible by these circulating agents. For the treatment of B cell Non-Hodgkin's lymphoma (NHL) RIT consists of the radiolabeled anti-CD20 mAbs ⁹⁰Y-ibritumomab tiuxetan (Zevalin) and ¹³¹I-tositumomab (Bexxar). Zevalin is now FDA approved as a late-stage add-on to the unlabeled anti-CD20 mAb Rituximab for the treatment of relapse and refractory NHL. Due to the high radiosensitivity of lymphomas only a relatively low absorbed dose is required to obtain an objective response. Although recent clinical trials have shown beneficial effect of the combination of Rituximab and Zevalin versus Rituximab alone (Tomblyn et al., 2012), Zevalin has only been approved for late-stage disease (patients with disease recurrence or non-responders to chemotherapy and immunotherapy with Rituximab).

RIT has its limitations. Due to the long blood half-life and circulation time of mAbs, the systemic administration of radiolabeled mAbs is characterized by a prolonged presence of radioactivity in blood and highly perfused organs. As an example, the ‘diagnostic’ SPECT scan performed several days after administration of ¹¹¹In-labeled Ibritumomab lacks sufficient specificity to accurately delineate CD20 positive lesions. In addition, myelotoxicity as a result of RIT is a well-known phenomenon and a dose-limiting factor (Emmanouilides et al., 2007). Furthermore, patients treated with Zevalin frequently suffer from neutropenia and thrombocytopenia.

The use of single domain antibodies, such as Nanobodies (Nbs), is an improvement to the toxicity problem of current radiolabeled mAb therapies, and TRNT using radiolabeled mAbs in general. Nbs are single domain antibodies with short blood half-life and superior characteristics compared to classical mAbs and their derived fragments for in vivo cell targeting (De Vos et al., 2013). In terms of molecular imaging of cancer, Nbs have been directed to a variety of membrane-bound cancer cell biomarkers, such as CEA, EGFR, HER2, and PSMA (D'Huyvetter et al., 2014). Because of their exceptional specificity of targeting, and the fact that they show to be functional after labeling with radionuclides, Nbs became valuable vehicles for nuclear imaging and TRNT (D'Huyvetter et al., 2014). Nevertheless, radiolabeled Nbs are characterized by significant retention in the kidneys after filtration from blood, which can lead to kidney related toxicities and kidney failure in case of being used as radiovehicles for TRNT.

In summary, the prior art teaches various toxicity problems of current radiolabeled anti-CD20 therapies. There is a need for radiolabeled CD20 binding agents that do not have the above mentioned limitations and thus have a lower retention in the kidneys while maintaining high therapeutic efficacy.

### SUMMARY

Applicants have generated and characterized human CD20 binding agents. Surprisingly, we found that a specific human CD20 binding agent showed low kidney retention, while retaining excellent in vivo tumor-targeting capacity.

It is an aspect of the present invention to provide a CD20 binding agent comprising three complementarity determining regions (CDR1, CDR2 and CDR3), wherein
(a) CDR1 comprises an amino acid sequence with at least 90% sequence identity with the amino acid sequence of SEQ ID No 1 or CDR1 comprises the amino acid sequence of SEQ ID No 1;
(b) CDR2 comprises an amino acid sequence with at least 90% sequence identity with the amino acid sequence of SEQ ID No 2 or CDR2 comprises the amino acid sequence of SEQ ID No 2;
(c) CDR3 comprises an amino acid sequence with at least 90% sequence identity with the amino acid sequence of SEQ ID No 3 or CDR3 comprises the amino acid sequence of SEQ ID No 3;
and wherein said CD20 binding agent is coupled to a radionuclide.

In one embodiment, the invention envisages a CD20 binding agent as described above that comprises a full length antibody or fragment thereof. In one embodiment, the invention envisages a CD20 binding agent as described above that comprises a single domain antibody.

Also envisaged is the CD20 binding agent as described above, for use in in vivo medical imaging, for use in the diagnosis and/or prognosis and/or prediction of treatment of cancer, for use as a medicine, for use in targeted radionuclide therapy, for use in the treatment of a disease or disorder involving cells expressing CD20 and for use in the treatment of cancer.

It is an aspect of the present invention to provide a nucleic acid comprising a nucleic acid sequence encoding an amino acid sequence comprising at least CDR1, CDR2 and CDR3 of the CD20 binding agent as described above. It is an aspect of the present invention to provide a vector comprising the nucleic acid as described above. It is an aspect of the present invention to provide a host cell comprising the nucleic acid or the vector as described above. It is also an aspect of the present invention to provide a pharmaceutical composition comprising the CD20 binding agent as described above in association with a pharmaceutically acceptable carrier.

It is an aspect of the present invention to provide an in vivo medical imaging method, the method comprising administering to a subject an effective amount of the CD20 binding agent as described above and detecting the CD20 binding agent in body areas of the subject. It is an aspect of the present invention to provide a diagnostic or prognostic method or a method for treatment prediction, the method comprising administering to a subject an effective amount of the CD20 binding agent as described above and detecting the CD20 binding agent in body areas of the subject.

Also envisaged is a method for treating a disease or disorder involving cells expressing CD20, the method comprising administering to a subject in need thereof a therapeutically effective amount of the CD20 binding agent as described above. In one embodiment, said disease or disorder involving cells expressing CD20 is cancer. It is an aspect of the present invention to provide a method for treating a disease or disorder involving cells expressing CD20, the method comprising selecting a subject on the basis of detection of CD20 on said cells and administering to said subject a therapeutic dose of the CD20 binding agent according to any of the above claims. In one embodiment, said disease or disorder involving cells expressing CD20 is cancer.

It is an aspect of the present invention to provide a kit comprising the CD20 binding agent as described above. Objects of the present invention will be clear from the description that follows.

### BRIEF DESCRIPTION OF DRAWINGS

FIG. 1 shows the binding specificity of anti-human CD20 Nanobodies (Nbs). Mean Fluorescence Intensity (MFI) is shown. Black bars indicate the MFI obtained from Nb incubation with CD20-positive Daudi cells, light grey bars show the MFI obtained from Nb incubation with CD20-negative Reh cells. The dark grey bar shows the negative control (NC), representing MFI generated by incubating detecting antibodies with Daudi cells, omitting Nb.

FIG. 2 shows the binding profile of anti-human CD20 Nbs on CD20-positive Daudi cells. Flow cytometry results indicate the MFI for six different dilutions of each Nb. Also indicated is the obtained half maximal effective concentration (EC50) for each Nb.

FIG. 3 shows the in vivo biodistribution of ^{99m}Tc-Nbs in a Daudi tumor xenografted mouse model.

Mean % IA/cm³ values of ^{99m}Tc-Nbs, including a control Nb (Ctrl Nb, targeting an epitope that is not present in these mice), are represented for the indicated organs/tissues in subcutaneous Daudi tumor-bearing mice.

FIG. 4 shows the in vivo biodistribution of ^{99m}Tc-Nbs in a hCD20+ B16 tumor model. Mean % IA/cm³ values of ^{99m}Tc-Nbs are represented for the indicated organs/tissues in subcutaneous hCD20-transfected B16 tumor-bearing mice.

FIG. 5 shows the ex vivo biodistribution of ^{99m}Tc-Nbs in a Daudi tumor xenografted mouse model. Mean % IA/g values of ^{99m}Tc-Nbs, including a control Nb (ctrl Nb, targeting an epitope that is not present in these mice), are represented for the indicated organs/tissues in subcutaneous Daudi tumor-bearing mice.

FIG. 6 shows the ex vivo biodistribution of ^{99m}Tc-Nbs in a hCD20+ B16 tumor model. Mean % IA/g values of ^{99m}Tc-Nbs, including a control Nb (ctrl Nb, targeting an epitope that is not present in these mice), are represented for the indicated organs/tissues in subcutaneous hCD20-transfected B16 tumor bearing mice.

FIG. 7 shows the tumor uptake of ^{99m}Tc-Nbs in a Daudi tumor model. Whereas a slightly higher tumor uptake was observed in mice injected with ^{99m}Tc-Nbs 9077, 9079, 9080 and 9081, no significant difference was observed between the different ^{99m}Tc-Nbs. Statistical analyses were conducted using the one-way ANOVA followed by a Bonferroni's multiple comparison tests and represented as mean±SD. The statistical difference in the figure is indicated as follows: * (p<0.05), ** (p<0.01), *** (p<0.001).

FIG. 8 shows the tumor uptake of ^{99m}Tc-Nbs in a hCD20+ B16 tumor model. Whereas a slightly higher tumor uptake was observed in mice injected with ^{99m}Tc-Nbs 9077, 9079 and 9081, no significant difference was observed between the different ^{99m}Tc-Nbs. Statistical analyses were conducted using the one-way ANOVA followed by a Bonferroni's multiple comparison tests and represented as mean±SD. The statistical difference in the figure is indicated as follows: * (p<0.05), ** (p<0.01), *** (p<0.001).

FIG. 9 shows the kidney uptake of ^{99m}Tc-Nbs in a Daudi tumor model. A significant lower kidney accumulation was observed in mice injected with ^{99m}Tc-Nb 9079. Statistical analyses were conducted using the one-way ANOVA followed by a Bonferroni's multiple comparison tests and represented as mean±SD. The statistical difference in the figure is indicated as follows: * (p<0.05), ** (p<0.01), *** (p<0.001).

FIG. 10 shows the in vitro characterization of ¹⁷⁷Lu-DTPA-anti-hCD20 Nbs 9077 and 9079. A) Binding specificity of ¹⁷⁷Lu-DTPA-anti-hCD20 Nb 9077 (A) and ¹⁷⁷Lu-DTPA-anti-hCD20 Nb 9079 (B). Specific competition with Rituximab was observed for both Nbs (p-values<0.0001 for both anti-hCD20 Nb). ¹⁷⁷Lu-DTPA-control Nb (C) showed significant lower, no specific, binding on hCD20^{pos} B16 cells, compared to ¹⁷⁷Lu-DTPA-anti-hCD20 Nb (p-values<0.0001 for both anti-hCD20 Nbs). B) and C) Affinity of ¹⁷⁷Lu-DTPA-Nb 9077 and ¹⁷⁷Lu-DTPA-Nb 9079 towards hCD20 receptor was obtained by incubating serial dilutions with hCD20^{pos} B16 cells. K_{D} values of 22.7±2.7 nM and 28.5±2.2 nM were obtained for ¹⁷⁷Lu-DTPA-anti-hCD20 Nbs 9077 and 9079, respectively. D) Internalization rate of ¹⁷⁷Lu-DTPA-anti-hCD20 Nb 9077 and ¹⁷⁷Lu-DTPA-anti-hCD20 Nb 9079 at different time points. E) Stability of ¹⁷⁷Lu-DTPA-Nb 9077 and ¹⁷⁷Lu-DTPA-Nb 9079 in human serum at 37° C. Still more than 91% of the radioactivity was protein-associated for both ¹⁷⁷Lu-DTPA-anti-hCD20 Nbs after 144 h.

FIG. 11 shows the in vivo characterization of ¹⁷⁷Lu-DTPA-anti-hCD20 Nbs 9077 and 9079. A) In vivo biodistribution of ¹⁷⁷Lu-DTPA-anti-hCD20 Nbs 9077 and 9079, and ¹⁷⁷Lu-DTPA-nontarget Nb (¹⁷⁷Lu-DTPA-ctrl Nb), co-infused with 150 mg/kg Gelofusin. micro-SPECT/CT images were obtained 1 h after i.v. injection of mice bearing hCD20^{pos} B16 tumors. B) Ex vivo biodistribution data obtained at 1.5 h p.i. Results are presented as mean % IA/g±SD (n=3 per Nb). Both ¹⁷⁷Lu-DTPA-anti-hCD20 Nbs showed significant higher tumor uptake compared to ¹⁷⁷Lu-DTPA-ctrl Nb (p<0.012), while no significant difference (ns) in tumor uptake was observed between ¹⁷⁷Lu-DTPA-Nb 9077 and ¹⁷⁷Lu-DTPA-Nb 9079. However, ¹⁷⁷Lu-DTPA-Nb 9079 showed significant lower kidney accumulation than ¹⁷⁷Lu-DTPA-Nb 9077 (p<0.0001).

FIG. 12 shows the dosimetry and therapeutic efficacy of ¹⁷⁷Lu-DTPA-Nb 9079 in hCD20^{pos} B16 tumor mouse model. A) Comparative dosimetry calculation of untagged ¹⁷⁷Lu-DTPA-Nb 9079 co-infused with 150 mg/kg Gelofusin versus ¹⁷⁷Lu-DTPA-Rituximab. Absorbed doses (in Gy) were extrapolated from the biodistribution data. B) Four group of mice (n=8 per group) received four i.v. injection of ¹⁷⁷Lu-DTPA-Nb 9079 (cumulative radioactive dose of 144±1.8 MBq), or ¹⁷⁷Lu-DTPA-ctrl Nb (cumulative radioactive dose of 135±2.74 MBq), Rituximab (200 μg/injection) or PBS. One group of mice received one i.v. injection of 7±1.48 MBq ¹⁷⁷Lu-DTPA-Rituximab. Tumor volumes were quantified using caliper measurements (mm³), in function of time (days). C) Resulting Kaplan-Meier survival curve. A significant difference in median survival was observed between mice treated with ¹⁷⁷Lu-DTPA-Nb 9079 and ¹⁷⁷Lu-DTPA-ctrl Nb (p<0.02) or PBS (p<0.001). No significant difference in median survival was observed between mice treated with ¹⁷⁷Lu-DTPA-Nb 9079, ¹⁷⁷Lu-DTPA-Rituximab, or Rituximab.

FIG. 13. Competition of Nb 9079 with Rituximab, Obinutuzumab and Ofatumomab for hCD20 receptor binding was analyzed by pre-incubating 5×10⁵ Daudi cells with a 100-fold molar excess of Rituximab, Obinutuzumab or Ofatumomab for 1 h at VC, prior to incubation with 1 μg/200 μL of Nb 9079. After washing, Nb binding was detected by incubating the cells with 2 μg Fluorescein IsoThioCyanate (FITC) labelled anti-HisTag Ab (Genscript) for 30 min at 4° C. Mean fluorescence intensity (MFI) was measured using LSR Fortessa Flow Cytometer (BD) and analysed with Flow Jo 7 software (Tree Star Inc., Ashland, Oreg., USA). Daudi cells unstained and Daudi cell+FITC Ab: background signal.

### DETAILED DESCRIPTION

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a”, “an” or “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2^{nd} ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

A “CD20 binding agent”, as used herein, is a protein-based agent capable of specific binding to CD20. In various embodiments, the CD20 binding agent may bind to the full-length and/or mature forms and/or isoforms and/or splice variants and/or fragments and/or any other naturally occurring or synthetic analogs, variants or mutants of CD20. In various embodiments, the CD20 binding agent of the invention may bind to any forms of CD20, including monomeric, dimeric, trimeric, tetrameric, heterodimeric, multimeric and associated forms. In an embodiment, the CD20 binding agent binds to the monomeric form of CD20. In another embodiment, the CD20 binding agent binds to a dimeric form of CD20. In another embodiment, the CD20 binding agent binds to a tetrameric form of CD20. In a further embodiment, the CD20 binding agent binds to the phosphorylated form of CD20, which may be either monomeric, dimeric, or tetrameric. In an embodiment, the present CD20 binding agent comprises an antigen binding site that comprises three complementarity determining regions (CDR1, CDR2 and CDR3). In an embodiment said antigen binding site recognizes one or more epitopes present on CD20. In various embodiments, the CD20 binding agent comprises a full length antibody or fragments thereof. In an embodiment, the CD20 binding agent comprises a single domain antibody. In a specific embodiment, the CD20 binding agent binds to CD20 of cynomolgus monkey (SEQ ID No 5, UniProt accession number NP_001274241). In a specific embodiment, the CD20 binding agent binds to human CD20 (SEQ ID No 4, UniProt accession number NP_690605).

In various embodiments, the CD20 binding agent comprises a sequence that is at least 60% identical to SEQ ID No 6. For example, the CD20 binding agent may comprise a sequence that is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% identical to SEQ ID No 6.

In various embodiments, the binding affinity of the CD20 binding agent of the invention for the full-length and/or mature forms and/or isoforms and/or splice variants and/or fragments and/or monomeric and/or dimeric and/or tetrameric forms and/or any other naturally occurring or synthetic analogs, variants, or mutants (including monomeric and/or dimeric and/or tetrameric forms) of human CD20 may be described by the equilibrium dissociation constant (K_{D}). In various embodiments, the CD20 binding agent binds to the full-length and/or mature forms and/or isoforms and/or splice variants and/or fragments and/or any other naturally occurring or synthetic analogs, variants, or mutants (including monomeric and/or dimeric and/or tetrameric forms) of human CD20 with a K_{D} of less than about 1 μM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, or about 5 nM, or about 2.5 nM, or about 1 nM.

In some embodiments, the CD20 binding agent described herein, includes derivatives that are modified, i.e. by the covalent attachment of any type of molecule to the CD20 binding agent such that covalent attachment does not prevent the activity of the agent. For example, but not by way of limitation, derivatives include CD20 binding agents that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc.

In various embodiments, the CD20 binding agent of the invention is multivalent, i.e. the CD20 binding agent comprises two or more antigen binding sites that recognize and bind one, two or more epitopes on the same antigen. In various embodiments, such multivalent CD20 binding agents exhibit advantageous properties such as increased avidity and/or improved selectivity. In an embodiment, the CD20 binding agent of the invention comprises two antigen binding sites and is biparatopic, i.e. binds and recognizes two different epitopes on the same antigen. In an embodiment, the CD20 binding agent of the invention comprises two antigen binding sites and is bivalent, i.e. binds and recognizes the same epitope on the same antigen. In a specific embodiment, the CD20 binding agent comprises one antigen binding site and is monovalent, i.e. binds and recognizes one epitope of CD20.

A first aspect of the present invention relates to a radiolabeled CD20 binding agent comprising three CDRs (CDR1, CDR2 and CDR3), wherein CDR1 comprises an amino acid sequence with at least 90% sequence identity with the amino acid sequence of SEQ ID No 1 or CDR1 comprises the amino acid sequence of SEQ ID No 1, CDR2 comprises an amino acid sequence with at least 90% sequence identity with the amino acid sequence of SEQ ID No 2 or CDR2 comprises the amino acid sequence of SEQ ID No 2 and CDR3 comprises an amino acid sequence with at least 90% sequence identity with the amino acid sequence of SEQ ID No 3 or CDR3 comprises the amino acid sequence of SEQ ID No 3. In the present application, CDRs are defined according to Kabat. Preferably, the CD20 binding agent is coupled to a radionuclide. In an embodiment, the CD20 binding agent is coupled or fused to the radionuclide either directly or through a coupling agent and/or a linker and/or a tag. In a specific embodiment, the CD20 binding agent is fused to the radionuclide via a His-tag. Methods used for radiolabeling the CD20 binding agent are conventional methods and are known to persons skilled in the art. Any available method and chemistry may be used for association or conjugation of the radionuclide to the CD20 binding agent. As an example, tricarbonyl chemistry may be used for radiolabeling (Xavier et al., 2012). In certain embodiments, the CD20 binding agent is coupled to a radionuclide that is damaging or otherwise cytotoxic to cells and the CD20 binding agent targets the radionuclide to CD20 expressing cells, preferentially to cancerous cell. The radiolabeled CD20 binding agent is used, for example—but not limited to—to target the damaging radionuclide to cancer tissue to preferentially damage or kill cancer cells.

As used herein, the term “radionuclide” relates to a radioactive label, which is a chemical compound in which one or more atoms have been replaced by a radioisotope. Radionuclides vary based on their characteristics, which include half-life, energy emission characteristics, and type of decay. This allows one to select radionuclides that have the desired mixture of characteristics suitable for use diagnostically and/or therapeutically. For example, gamma emitters are generally used diagnostically and alpha and beta emitters are generally used therapeutically. However, some radionuclides are both gamma emitters, alpha emitters and/or beta emitters, and thus, may be suitable for both uses. Radionuclides, as used herein, include for example—but not limited to—Actinium-225, Astatine-209, Astatine-210, Astatine-211, Bismuth-212, Bismuth-213, Brome-76, Caesium-137, Carbon-11, Chromium-51, Cobalt-60, Copper-64, Copper-67, Dysprosium-165, Erbium-169, Fermium-255, Fluorine-18, Gallium-67, Gallium-68, Gold-198, Holium-166, Indium-111, Iodine-123, Iodine-124, Iodine-125, Iodine-131, Iridium-192, Iron-59, Krypton-81m, Lead-212, Lutetium-177, Molydenum-99, Nitrogen-13, Oxygen-15, Palladium-103, Phosphorus-32, Potassium-42, Radium-223, Rhenium-186, Rhenium-188, Samarium-153, Technetium-99m, Radium-223, Rubidium-82, Ruthenium-106, Sodium-24, Strontium-89, Terbium-149, Thallium-201, Thorium-227, Xenon-133, Ytterbium-169, Ytterbium-177, Yttrium-86, Yttrium-90, Zirconium-89. In certain embodiments, the radionuclide is selected from the group of radionuclides as described above. In a specific embodiment, the radionuclide is selected from the group consisting of Technetium-99m, Gallium-68, Fluorine-18, Indium-111, Zirconium-89, Iodine-123, Iodine-124, Iodine-131, Astatine-211, Bismuth-213, Lutetium-177 and Yttrium-86.

According to particular embodiments, the CD20 binding agent as described above comprises a full length antibody or fragment thereof. According to further particular embodiments said CD20 binding agent comprises a single domain antibody. As used herein, the term “single domain antibody” defines molecules wherein the antigen binding site is present on, and formed by, a single immunoglobulin domain (which is different from conventional immunoglobulins or their fragments, wherein typically two immunoglobulin variable domains interact to form an antigen binding site). It should however be clear that the term “single domain antibody” does comprise fragments of conventional immunoglobulins wherein the antigen binding site is formed by a single variable domain. Generally, an immunoglobulin single variable domain will be an amino acid sequence comprising 4 framework regions (FR1 to FR4) and 3 complementary determining regions (CDR1 to CDR3), preferably according to the following formula (1): FR1-CDR1-FR2-CDR2-FR3-CDR3-FR4 (1), or any suitable fragment thereof (which will then usually contain at least some of the amino acid residues that form at least one of the CDRs). Single domain antibodies comprising 4 FRs and 3 CDRs are known to the person skilled in the art and have been described, as a non-limiting example, in Wesolowski et al. 2009. In a specific embodiment, the single domain antibody as described herein is a Nanobody or VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH2 and CH3). VHHs are commercially available under the trademark of NANOBODIES. In some embodiments, the single domain antibody as described herein is an immunoglobulin single variable domain or ISVD.

According to particular embodiments, the CD20 binding agent as described above is useful for in vivo medical imaging. As used herein, the term “in vivo medical imaging” refers to the technique and process that is used to visualize the inside of an organism's body (or parts and/or functions thereof), for clinical purposes (e.g. disease diagnosis, prognosis or therapy monitoring) or medical science (e.g. study of anatomy and physiology). Examples of medical imaging methods include invasive techniques, such as intravascular ultrasound (IVUS), as well as non-invasive techniques, such as magnetic resonance imaging (MRI), ultrasound (US) and nuclear imaging. Examples of nuclear imaging include positron emission tomography (PET) and single photon emission computed tomography (SPECT). In a preferred embodiment, a nuclear imaging approach is used for in vivo medical imaging. According to one specific embodiment, in vivo pinhole SPECT/micro-CT (computed tomography) imaging is used as in vivo imaging approach.

According to particular embodiments, the CD20 binding agent as described above is useful for targeted radionuclide therapy. “Targeted radionuclide therapy”, as used herein, refers to the targeted delivery of a radionuclide to a disease site and the subsequent damage of the targeted cells and adjacent cells (bystander effect). In targeted radio-therapy, also referred to as systemic targeted radionuclide therapy (STaRT), the biological effect is obtained by energy absorbed from the radiation emitted by the radionuclide. Non-limiting exemplary radionuclides are Iodine-131, Astatine-211, Bismuth-213, Lutetium-177 or Yttrium-86. Exemplary radionuclides that can be used to damage cells, such as cancer cells, are high energy emitters. For example, a high energy radionuclide is selected and targeted to cancer cells. The high energy radionuclide preferably acts over a short range so that the cytotoxic effects are localized to the targeted cells. In this way, radio-therapy is delivered in a more localized fashion to decrease damage to non-cancerous cells.

The present invention also pertains to the use of the CD20 binding agent as described above for disease diagnosis and/or prognosis and/or treatment prediction in a subject. As non-limiting example, a subject having cancer or prone to it can be determined based on the expression levels, patterns, or profile of CD20 in a test sample from the subject compared to a predetermined standard or standard level in a corresponding non-cancerous sample. In other words, CD20 polypeptides can be used as markers to indicate the presence or absence of cancer or the risk of having cancer, as well as to assess the prognosis of the cancer and for prediction of the most suitable therapy.

Also envisaged is the use of said CD20 binding agent as a medicine and the use of the CD20 binding agent as described above in the treatment of a disease or disorder involving cells expressing CD20. Non-limiting examples of diseases or disorders involving cells expressing CD20 are auto-immune diseases such as rheumatoid arthritis (RA), juvenile rheumatoid arthritis, systemic lupus erythematosus (SLE), vasculitis, Wegener's disease, inflammatory bowel disease, idiopathic thrombocytopenic purpura (ITP), thrombotic thrombocytopenic purpura (TTP), autoimmune thrombocytopenia, multiple sclerosis (MS), chronic inflammatory demyelinating polyneuropathy, psoriasis, IgA nephropathy, IgM polyneuropathies, myasthenia gravis, diabetes mellitus, Reynaud's syndrome, Crohn's disease, ulcerative colitis, gastritis, Hashimoto's thyroiditis, ankylosing spondylitis, hepatitis C-associated cryoglobulinemic vasculitis, chronic focal encephalitis, hemophilia A, membranoproliferative glomerulonephritis, adult and juvenile dermatomyositis, adult polymyositis, chronic urticaria, primary biliary cirrhosis, neuromyelitis optica, Graves' dysthyroid disease, bullous skin disorders, bullous pemphigoid, pemphigus, Churg-Strauss syndrome, asthma, psoriatic arthritis, dermatitis, respiratory distress syndrome, meningitis, encephalitits, anti-NMDA receptor encephalitis, uveitis, eczema, atherosclerosis, leukocyte adhesion deficiency, juvenile onset diabetes, Reiter's disease, Behcet's disease, hemolytic anemia, atopic dermatitis, Wegener's granulomatosis, Omenn's syndrome, chronic renal failure, acute infectious mononucleosis, HIV and herpes-associated disease, systemic sclerosis, Sjorgen's syndrome and glomerulonephritis, dermatomyositis, ANCA vasculitis, aplastic anemia, autoimmune anemia, autoimmune hemolytic anemia (AIHA), pure red cell aplasia, Evan's syndrome, factor VIII deficiency, hemophilia A, autoimmune neutropenia, Castleman's syndrome, Goodpasture's syndrome, solid organ transplant rejection, graft versus host disease (GVHD), autoimmune hepatitis, lymphoid interstitial pneumonitis (HIV), bronchiolitis obliterans (non-transplant), Guillain-Barre Syndrome, large vessel vasculitis, giant cell (Takayasu's) arteritis, medium vessel vasculitis, Kawasaki's Disease, polyarteritis nodosa, Devic's disease, autoimmune pancreatitis, Opsoclonus Myoclonus Syndrome (OMS), IgG4-related disease, scleroderma and chronic fatigue syndrome

In another aspect, the present invention envisages the use of the CD20 binding agent as described above in the treatment of cancer. Non-limiting examples of cancer are melanoma, non-Hodgkin's lymphoma (NHL), lymphocyte predominant subtype of Hodgkin's lymphoma, precursor B cell lymphoblastic leukemia/lymphoma, mature B cell neoplasm, B cell chronic lymphocytic leukemia (CLL), small lymphocytic lymphoma (SLL), B cell prolymphocytic leukemia, lymphoplasmacytic lymphoma, mantle cell lymphoma (MCL), follicular lymphoma (FL) including low-grade, intermediate-grade and high-grade FL, cutaneous follicle center lymphoma, marginal zone B cell lymphoma, MALT type marginal zone B cell lymphoma, nodal marginal zone B cell lymphoma, splenic type marginal zone B cell lymphoma, hairy cell leukemia, diffuse large B cell lymphoma, Burkitt's lymphoma, plasmacytoma, plasma cell myeloma, post-transplant lymphoproliferative disorder, Waldenstrom's macroglobulinemia, multiple myeloma and anaplastic large-cell lymphoma (ALCL).

In another aspect, a nucleic acid comprising a nucleic acid sequence coding at least for CDR1, CDR2 and CDR3 of the above described CD20 binding agent is envisaged. As used herein, the term “nucleic acid” refers to a polymeric form of nucleotides of any length, either deoxyribonucleotides or ribonucleotides, or analogs thereof. Nucleic acids may have any three-dimensional structure, and may perform any function, known or unknown. Non-limiting examples of nucleic acids include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

In another aspect, a vector comprising the above described nucleic acid is envisaged. The term “vector” refers to a nucleic acid assembly capable of transferring gene sequences to target cells (e.g. viral vectors, non-viral vectors, particulate carriers, and liposomes). The term “expression vector” refers to a nucleic acid assembly containing a promoter which is capable of directing the expression of a sequence or gene of interest in a cell. Vectors typically contain nucleic acid sequences encoding selectable markers for selection of cells that have been transfected by the vector. Generally, “vector construct,” “expression vector,” and “gene transfer vector,” refer to any nucleic acid construct capable of directing the expression of a gene of interest and which can transfer gene sequences to target cells. Thus, the term includes cloning and expression vehicles, as well as viral vectors.

In another aspect, a host cell comprising the above described nucleic acid or vector is envisaged. Suitable host cells include E. coli, Saccharomyces cerevisiae, Schizosaccharomyces pombe, Pichia pastoris, and the like. Suitable animal host cells include HEK 293, COS, S2, CHO, NSO, DT40 and the like. The cloning, expression and/or purification of the immunoglobulin single variable domains can be done according to techniques known by the skilled person in the art.

In a further related aspect, the disclosure contemplates a pharmaceutical composition comprising the CD20 binding agent as described above, in association with a pharmaceutically acceptable carrier. Therefore, the radiolabeled CD20 binding agent may be formulated in a physiologically or pharmaceutically acceptable carrier suitable for in vivo administration. In certain embodiments, such compositions are suitable for oral, intravenous or intraperitoneal administration. In other embodiments, such compositions are suitable for local administration directly to the site of a tumor. In certain embodiments, such compositions are suitable for subcutaneous administration.

In another aspect, the disclosure provides an in vivo medical imaging method. The method comprises administering to a subject, such as a human or non-human subject, an effective amount of the radiolabeled CD20 binding agent, as described herein. The effective amount is the amount sufficient to label the desired cells and tissues so that the labeled structures are detectable over the period of time of the analysis. The method further comprises collecting one or more images of the subject and displaying the one or more images of the subject. The images may be taken over a period of time, including multiple images over a period of time. The collecting and displaying of said images is done by a commercially available scanner and the accompanying computer hardware and software. For example PET and SPECT scanners may be used. Moreover, to further improve the usefulness of the images generated, CT, X-ray or MRI may be simultaneously or consecutively used to provide additional information, such as depiction of structural features of the subject. For example, dual PET/CT scanners can be used to collect the relevant data, and display images that overlay the data obtained from the two modalities. Any of the radionuclides suitable for in vivo imaging and the corresponding radiolabeled agents can be used in these methods. By way of example, when selecting a radionuclide for in vivo imaging, a gamma or positron emitting radionuclide or a radionuclide that decays by electron transfer may be preferred. Emissions can then be readily detected using, for example, positron emission tomography (PET) or single photon emission computed tomography (SPECT). Generally, it is desirable that the half-life of the radionuclide is long enough to be made and used in testing, but not so long that radioactivity lingers in the patient for a considerable period of time after the test has been performed. Moreover, the amount of radioactivity used to label can be modulated so that the minimum amount of total radiation is used to achieve the desired effect.

In various embodiments, the pharmaceutical composition of the present invention is co-administered in conjunction with additional therapeutic agent(s). Co-administration can be simultaneous or sequential.

In one embodiment, the additional therapeutic agent and the CD20 binding agent of the present invention are administered to a subject simultaneously. Simultaneously means that the additional therapeutic agent and the CD20 binding agent are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the CD20 binding agent can be by simultaneous administration of a single formulation (e.g. a formulation comprising the additional therapeutic agent and the CD20 binding agent) or of separate formulations (e.g. a first formulation including the additional therapeutic agent and a second formulation including the CD20 binding agent).

Co-administration does not require the therapeutic agents to be administered simultaneously, if the timing of their administration is such that the pharmacological activities of the additional therapeutic agent and the CD20 binding agent overlap in time, thereby exerting a combined therapeutic effect. For example, the additional therapeutic agent and the CD20 binding agent can be administered sequentially. The term “sequentially” as used herein means that the additional therapeutic agent and the CD20 binding agent are administered with a time separation of more than about 60 minutes. For example, the time between the sequential administration of the additional therapeutic agent and the CD20 binding agent can be more than about 60 minutes, more than about 2 hours, more than about 5 hours, more than about 10 hours, more than about 1 day, more than about 2 days, more than about 3 days, more than about 1 week, or more than about 2 weeks, or more than about one month apart. The optimal administration times will depend on the rates of metabolism, excretion, and/or the pharmacodynamic activity of the additional therapeutic agent and the CD20 binding agent being administered. Either the additional therapeutic agent or the CD20 binding agent cell may be administered first.

Co-administration also does not require the therapeutic agents to be administered to the subject by the same route of administration. Rather, each therapeutic agent can be administered by any appropriate route, for example, parenterally or non-parenterally.

In some embodiments, the CD20 binding agent described herein acts synergistically when co-administered with another therapeutic agent. In such embodiments, the CD20 binding agent and the additional therapeutic agent may be administered at doses that are lower than the doses employed when the agents are used in the context of monotherapy.

In some embodiments, the present invention pertains to chemotherapeutic agents as additional therapeutic agents. For example, without limitation, such combination of the present CD20 binding agents and chemotherapeutic agent find use in the treatment of cancers, as described elsewhere herein. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; def of amine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of photodynamic therapy.

In some embodiments, the present invention relates to combination therapy with one or more immune-modulating agents, for example, without limitation, agents that modulate immune checkpoint. In various embodiments, the immune-modulating agent targets one or more of PD-1, PD-L1, and PD-L2. In various embodiments, the immune-modulating agent is PD-1 inhibitor. In various embodiments, the immune-modulating agent is an antibody specific for one or more of PD-1, PD-L1, and PD-L2. For instance, in some embodiments, the immune-modulating agent is an antibody such as, by way of non-limitation, nivolumab, (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), MPDL328OA (ROCHE). In some embodiments, the immune-modulating agent targets one or more of CD137 or CD137L. In various embodiments, the immune-modulating agent is an antibody specific for one or more of CD137 or CD137L. For instance, in some embodiments, the immune-modulating agent is an antibody such as, by way of non-limitation, urelumab (also known as BMS-663513 and anti-4-1BB antibody). In some embodiments, the present chimeric protein is combined with urelumab (optionally with one or more of nivolumab, lirilumab, and urelumab) for the treatment of solid tumors and/or B-cell non-Hodgkins lymphoma and/or head and neck cancer and/or multiple myeloma. In some embodiments, the immune-modulating agent is an agent that targets one or more of CTLA-4, AP2M1, CD80, CD86, SHP-2, and PPP2R5A. In various embodiments, the immune-modulating agent is an antibody specific for one or more of CTLA-4, AP2M1, CD80, CD86, SHP-2, and PPP2R5A. For instance, in some embodiments, the immune-modulating agent is an antibody such as, by way of non-limitation, ipilimumab (MDX-010, MDX-101, Yervoy, BMS) and/or tremelimumab (Pfizer). In some embodiments, the present chimeric protein is combined with ipilimumab (optionally with bavituximab) for the treatment of one or more of melanoma, prostate cancer, and lung cancer. In various embodiments, the immune-modulating agent targets CD20. In various embodiments, the immune-modulating agent is an antibody specific CD20. For instance, in some embodiments, the immune-modulating agent is an antibody such as, by way of non-limitation, Ofatumumab (GENMAB), obinutuzumab (GAZYVA), AME-133v (APPLIED MOLECULAR EVOLUTION), Ocrelizumab (GENENTECH), TRU-015 (TRUBION/EMERGENT), veltuzumab (IMMU-106). In an embodiment, the immune modulating agent is an antibody that targets OX40.

A “subject”, as used herein, also refers to organisms which are within the class mammalia, including dogs, cats, mice, guinea pigs, rats, rabbits, humans, chimpanzees, monkeys, etc. In preferred embodiments, the subjects will be humans. In certain embodiments, the subject is a patient having or suspected of having a disease or disorder involving cells expressing CD20, and the in vivo medical imaging method is used to help diagnose and/or prognose the presence of the disease or disorder. In certain embodiments, the subject is a patient having or suspected of having cancer, and the in vivo medical imaging method is used to help diagnose and/or prognose the presence and location of the cancer. In certain embodiments, the in vivo medical imaging method is used to follow a patient's progression over time (e.g. over the course of treatment). In certain embodiments, the patient has or is suspected of having leukemia. In a specific embodiment, the patient has or is suspected of having CD20 positive lymphoma. The terms “patient”, “individual” and “subject” are used interchangeably herein, and cover mammals including humans. The term does not denote a particular age or sex. Thus, adult and newborn subjects, as well as fetuses, whether male or female, are intended to be included within the scope of this term. In a specific embodiment, the term “subject” refers to a human individual.

As used herein, the terms “diagnosis”, “prognosis” and/or “prediction” comprise diagnosing, prognosing and/or predicting a certain disease and/or disorder, thereby predicting the onset and/or presence of a certain disease and/or disorder, and/or predicting the progress and/or duration of a certain disease and/or disorder, and/or predicting the response of a patient suffering from a certain disease and/or disorder to therapy.

In another aspect, the present invention provides a diagnostic and/or prognostic and/or predictive method, the method comprising administering to a subject the CD20 binding agent as described above and detecting the CD20 binding agent in body areas such as—but not limited to—the head and neck, thorax and abdomen of the subject. Said detection may be done by the above described in vivo medical imaging methods.

“Treatment” and “treating,” as used herein refer to therapeutic treatment, wherein the objective is to inhibit or slow down (lessen) the targeted disorder (e.g. cancer) or symptom of the disorder, or to improve a symptom, even if the treatment is partial or ultimately unsuccessful. Those in need of treatment include those already diagnosed with the disorder as well as those prone or predisposed to contract the disorder or those in whom the disorder is to be prevented. For example, in tumor (e.g. cancer) treatment, a therapeutic agent can directly decrease the pathology of tumor cells, or render the tumor cells more susceptible to treatment by other therapeutic agents or by the subject's own immune system.

In another aspect, the disclosure provides a method for treating a disease or disorder involving cells expressing CD20, the method comprising administering to a patient in need thereof a therapeutically effective amount of the CD20 binding agent as described above. Therefore, the CD20 binding agent is labeled with a high energy emitting radionuclide which is targeted to CD20 expressing cells to damage said cells.

In another aspect, the present invention provides a method of treating cancer, the method comprising administering to a patient in need thereof a therapeutically effective amount of the CD20 binding agent as described above. Therefore, the CD20 binding agent is labeled with a high energy emitting radionuclide which is targeted to cancerous cells to damage said cells.

As used herein, the term “therapeutically effective amount” means the amount needed to achieve the desired result or results when used in therapy.

In another aspect, the present invention also provides kits for the administration of any CD20 binding agent described herein. The kit is an assemblage of materials or components, including the inventive CD20 binding agent or the pharmaceutical composition described herein. The exact nature of the components configured in the kit depends on its intended purpose. In one embodiment, the kit is configured for the purpose of treating human subjects. In one embodiment the kit comprises a solid support.

Instructions for use may be included in the kit. Instructions for use typically include a tangible expression describing the technique to be employed in using the components of the kit to effect a desired therapeutic outcome, such as to treat cancer. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner stored in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label which indicates the contents and/or purpose of the kit and/or its components.

In another aspect, the present invention also provides a solid support comprising the CD20 binding agent.

### EXAMPLES

### Materials and Methods to the Examples 1-7

Cloning of the Human CD20 (hCD20) Gene

Human CD20 was amplified from the Orfeome v5.1 collection (ID 11051) with forward primer 5′-GATAAGATCTCAGGCGGATCCACAACACCCAGAAATTCAG (0-7954) and reverse primer 5′-GGTTTTTTCTCTAGATCAAGGAGAGCTGTCATTTTCTATTGG (0-7956). The amplified product was cut with BglII and XbaI and ligated into the mammalian expression vector pMet7. The plasmid was used for transient transfection of Hek293T cells and for the generation of CHO-K1 and B16-F10 clones stably expressing human CD20.

Generation of hCD20-Specific Nanobodies

A VHH library was subject to 3 consecutive rounds of panning (in solution), performed on stably transfected CHO-K1 cells expressing human CD20. A parallel panning was performed on parental (non-transfected CHO-K1) cells to serve as negative control for the calculation of CD20-specific phage enrichment. The enrichment for antigen-specific phages was assessed after each round of panning by comparing the number of phagemid particles eluted from transfected cells with the number of phagemid particles eluted from parental cells. These experiments suggested that the phage population was enriched (for antigen-specific phages) about 2-, 8- and 4-fold after 1^{st}, 2^{nd} and 3^{rd} rounds of panning, respectively. In total of 95 colonies from the 2^{nd} round of panning were randomly selected and their crude periplasmic extracts (including soluble Nanobodies) were analyzed by cell ELISA for specific binding to CD20 transfected CHO-K1, as compared to parental cells.

Subcloning of hCD20-Specific Nanobody Sequences

The Nanobody gene cloned in the pMECS vector contains the PelB signal sequence at the N-terminus and a HA tag and a His₆ tag at the C-terminus (PelB leader-Nanobody-HA-His₆). The PelB leader sequence directs the Nanobody to the periplasmic space of E. coli and the HA and His₆ tags can be used for the Nanobody purification and detection. Upon production from the pMECS vector, the His₆ tag is cleaved off upon storage or upon ^{99m}Tc-labeling. Therefore the Nanobody gene was subcloned from pMECS into pHEN6 vector by the use of the PstI and BstEII restriction sites and transformed in competent E. coli WK6 cells. The Nanobody gene cloned in the pHEN6 vector contains the PelB signal sequence at the N-terminus and His₆-tail at the C-terminus. The PelB leader sequence directs the Nanobody to the periplasmic space of E. coli and the His-tag can be used for the purification and detection of Nanobody, as well as for ^{99m}Tc-labeling.

Expression and Purification of hCD20-Specific Nanobodies
Production Via pHEN6 Expression Vector

E. coli WK6 cells were transformed with pHEN6 expression vector and plated out on LB agar plates supplemented with 100 μg/mL ampicillin and 2% glucose and incubated overnight at 37° C. After that, a starter culture was prepared by inoculation of a single colony from LB agar plate with a sterile tip in 15 ml LB+100 μg/mL ampicillin following overnight incubation at 37° C., shaking at 200 rpm (New Brunswick Incubator Shaker). Next, 1 ml of the starter culture was inoculated in the 330 ml TB baffled shaker flasks supplemented with 100 μg/mL ampicillin and 0.1% glucose, and then incubated at 37° C. while shaking at 200 rpm till 0.6<OD600 nm<0.9 was reached. Hereafter, the expression of Nbs in E. coli WK6 periplasm was induced by adding IPTG (1 mM final concentration), following overnight incubation at 28° C., shaking at 200 rpm in a New Brunswick incubator shaker.

### Periplasmic Extraction

Upon expression, the periplasmic extracts containing the His-tagged Nbs were extracted by osmotic shock. Briefly, the bacterial pellets were obtained and resuspended with TES (4 ml TES per pellet from 330 ml culture), following shaking at 200 rpm for 1 h on ice. After that an osmotic shock was performed by adding 8 ml TES/4 (per pellet from 330 ml culture) to the mixture followed by incubation for 2 h on ice while shaking at 200 rpm. Finally, the mixture was centrifuged and the supernatant containing the periplasmic extract was collected.

### Immobilized Metal Affinity Chromatography (IMAC)

The His-tagged Nbs were purified from the periplasmic extract by IMAC using nickel beads. Briefly, 1 mL of HIS-Select Nickel Gel solution (1 mL/L of culture; Sigma-Aldrich) was directly added into the falcon tube containing the periplasmic extract and incubated for 1 h while shaking at 200 rpm at room temperature. The periplasmic extract was then centrifuged and the supernatant was discarded. The periplasmic-HIS-Select pellets were then washed with PBS and loaded into the PD-10 column. HIS-Select column was washed by pipetting 20 mL PBS per mL HIS-Select solution into the column and then letting the PBS buffer drain by gravitational force. Nb fractions were eluted with 10 mL of 0.5 M Imidazole in PBS and the OD₂₈₀ₙₘ was measured using Nanodrop™ (Isogen Life Sciences ND 10000). Eluted Nbs were stored at 4° C. and later used for size exclusion chromatography (SEC).

### Size Exclusion Chromatography (SEC)

Following IMAC-purification, the produced Nbs were further purified using SEC. This technique separates molecules based on their molecular weight. Therefore, the concentrated IMAC pooled fraction was loaded onto a Hiload S75 column that was attached to AKTA Express System. During the run, the proteins were collected in 96 well collection plate and the fraction having a high peak on the curve relative to the expected molecular weight of a Nb were put together in a 50 ml tube and the final concentrations of these proteins were measured. Nbs were then stored at 4° C. or −20° C. until further usage.

### In Vitro Targeting

In vitro targeting of hCD20 receptor by Nbs was evaluated by performing flow cytometry. Cells were collected, washed and counted. For each condition, 5×10⁵ cells were used. Briefly, Daudi and Reh cells were incubated with 1 μg of Nb in 200 μl FACS buffer (PBS containing 1% bovine serum albumin (BSA; Thermo Fisher Scientific—Perbioscience) and 0.02% sodium azide (NaN₃; Acros) for 1 h at 4° C. After washing with ice-cold FACS buffer, the Nb binding was detected by incubating the cells with 2 μg Fluorescein-IsoThioCyanate (FITC) labeled anti-His-Tag antibody (Genscript) in 20 μl FACS buffer, for 30 min at 4° C. Background controls included: Daudi cells unstained, Daudi cells incubated with 2 μg FITC labelled anti-His-Tag antibody and Daudi cells incubated with 2 μg Anti-IgG1 PE human.

### EC50 Determination of Nbs

The half maximal effective concentration (EC50) for binding to hCD20 was analysed for each Nb by performing flow cytometry using 6 different dilutions (1000 nM, 200 nM, 40 nM, 8 nM, 1.5 nM and 0.5 nM) of each Nb. Each Nb dilution in 200 μl FACS buffer was incubated with 5×10⁵ Daudi cells for 1 h at 4° C. After washing with ice-cold FACS buffer, the Nb binding was detected by incubating each condition with FITC labelled anti-His-Tag antibody in 20 μl FACS buffer, for 30 min at 4° C.

### Cell Lines and Culture Conditions

The human Burkitt's lymphoma cell line Daudi (hCD20⁺) and human acute lymphocytic leukemia cell line REH (hCD20⁻) were obtained from American Type Culture Collection (ATCC, Manassas, Va., USA). The hCD20⁺ transfected mouse B16 cell line (hCD20⁺ B16) was generated by stable integration of a hCD20 expression vector using methods known in the art. Daudi and Reh cell lines were cultured using RPMI-1640 medium supplemented with 10% heat-inactivated FBS, 2 mM L-glutamine and 0.1 mg/ml streptomycin. The hCD20⁺ B16 cell line was cultured in DMEM supplemented with 10% heat-inactivated FBS, 2 mM L-glutamine and 0.1 mg/ml streptomycin. All media and supplements were obtained from Life Technologies (Paisley, UK). Cells were grown at 37° C. in a humidified atmosphere with 5% CO₂. Prior to use for in vitro or in vivo experiments, hCD20⁺ B16 cells were detached with trypsin-EDTA in PBS (Paisley, UK).

### Animal Models

Male CB17 SCID mice and female C57BL/6 mice (Charles River, Wilmington, Mass.) at ages six to twelve weeks were used. CB17 SCID mice were injected subcutaneously (s.c.) in the right hind limb with 12×10⁶ Daudi cells in PBS, while C57BL/6 mice were injected s.c. in the right hind limb with 1×10⁶hCD20⁺ B16 cells in PBS. Prior to the s.c. injection of cells, all mice were sedated with 2.5% isoflurane (Abbott, Ottignies-Louvain-la Neuve, Belgium). Tumors were allowed to grow up to 250-300 mm³. All experiments were approved by the ‘Ethical Committee for Animal Experiments’ of the Vrije Universiteit Brussel and performed according to the national and European guidelines and regulations.

Radiolabelling of Nbs with Technetium-99m (^{99m}Tc)

Nbs were radiolabeled with ^{99m}Tc at their His-tag using straightforward tricarbonyl chemistry (Xavier et al., 2012). Briefly, ^{99m}Tc-Tricarbonyl precursor (^{99m}Tc(CO)3)H2O)3)⁺) was prepared by adding 1 mL of the ^{99m}TcO₄⁻ solution (⁹⁹Mo/^{99m}Tc generator eluate, Drytec, GE Healthcare; maximum 100 mCi) to the IsoLink™ kit (4.5 mg sodium baronocarbonate, 2.85 mg sodium tetraborate, 8.5 mg sodium titrate, 7.15 mg sodium carbonate; Covidien, St Louis, USA). After incubation of this mixture at 100° C. for 20 min, the reaction mixture was cooled in water and the pH adjusted to 7.4 by adding 1 M HCl. After that, the His-tagged Nbs were then labelled with ^{99m}Tc-Tricarbonyl by mixing 50 μg (in 50 μL PBS) of each Nb with 500 μl of ^{99m}Tc-Tricarbonyl at pH 7.4. After incubating the mixture at 50° C. for 60-90 min, the ^{99m}Tc-Nbs were then separated from the free ^{99m}Tc-Tricarbonyl and ^{99m}TcO4⁻ by size exclusion using the NAP-5 column (Sephadex, GE Helathcare). Lastly, the NAP-5 eluate was passed through a 0.22 μm membrane filter (Millex, Millipore) to eliminate possible aggregates and the radiochemical purity was evaluated by instant Thin Layer Chromatography (iTLC SG, Pall Corporation, Belgium).

In Vivo Targeting of ^{99m}Tc-Labeled Nbs

The biodistribution and in vivo targeting capacity of all 15 Nbs was analysed in two tumor models, namely Daudi and hCD20⁺ B16 tumor models. For Daudi tumors, 12×10⁶ Daudi cells per mouse were required and about 5 weeks for tumor development. For hCD20⁺ B16 tumors, 1×10⁶ hCD20⁺ B16 cells were required and about 1 week for tumor development. An average of 3 mice with weight of 25±5 g were injected with a single Nb. The mice were anaesthetised with inhalational anesthetic (Isofluorane) prior to i.v. injection of 100-200 μL, 45-155 MBq (about 5-10 μg), ^{99m}Tc-Tricarbonyl-Nbs. Approximately 1 h post-injection of Nbs, mice were prepared for performing SPECT/micro-CT scans by i.p. injection of medetomidine-ketamine solution (18.75 mg/kg of mice weight ketamine hydrochloride (Ketamine 1000, CEVA, Brussel, Belgium) and 0.5 mg/kg medetomidine hydrochloride (Domitor, Pfizer, Brussel, Belgium).

### In Vivo Pinhole SPECT/Micro-CT Imaging Studies

SPECT/micro-CT scans (using gamma rays and x-rays, respectively) were performed on each mouse, 1 h after injection of ^{99m}Tc-labeled Nbs. The micro-CT scan (Skyscan 1178, Skyscan) was performed in order to obtain anatomical three-dimensional (3D) images (50 KeV, 615 mA, rotation 360°). The distribution of radiolabeled Nbs was detected by performing pinhole SPECT scans using a dual-heady-camera (e.cam 180, Siemens) with two multi-pinhole collimators, with three 1.5 mm pinholes in each collimator (200 mm focal length and 80 mm radius of rotation). After reconstruction of all images, image viewing and quantification was performed using ‘A Medical Image Data Examiner’ (AMIDE) software. Ellipsoid regions of interest (ROIs) were drawn around total body, the tumor and also organs and tumor, based on the CT images. For the tumor delineation, a threshold of 80% of the maximum pixel values on the SPECT images was chosen. Uptake of the radiolabeled Nb was calculated as the radioactive signal in tissues divided by the total injected activity, normalized for the region of interest and presented as % IA/cm³.

### Ex Vivo Biodistribution Studies

30 min after SPECT/micro-CT, mice were euthanized and dissected with harvesting of different organs, tissues and tumors. After that, organs, tissues and tumors were weighed and the associated radioactivity per organ/tissue was measured using a gamma counter (Cobra II Inspector 5003, Canberra Packard, USA). The results were expressed as percentage of injected activity per gram of tissue (% IA/g).

### Materials and Methods to the Examples 8-11

### Production and Purification of Selected Anti-hCD20 Nbs

DNA fragments encoding for anti-hCD20 Nbs 9077 and 9079 were recloned in either pHEN6 expression vector that encodes for a carboxyterminal hexahistidine tail (His-tag), or in the pHEN21 expression vector that does not encode for a carboxyterminal amino acid (AA) tail, and subsequently produced in E. coli WK6 cultures. The non-targeting R3B23 Nb, referred to as ctrl Nb, was produced similarly and used as negative control in all experiments. The expression of Nbs was induced overnight at 28° C. with 1 mM isopropyl-β-D-thiogalactoside (IPTG). Periplasmic extracts, containing the soluble fragments, were obtained by osmotic shock. His-tagged Nbs were further purified using immobilized metal affinity chromatography on His-Select Nickel Affinity Gel. Untagged Nbs were further purified on a protein A column (Sigma St Louis, Mo., USA) and reconstituted in PBS via size-exclusion chromatography using a Superdex 75 16/60 column (GE Healthcare Biosciences, Pittsburgh, Pa., USA) equilibrated in PBS.

In Vitro Competition of Nb 9079 with Rituximab, Obinituzumab and Ofatumomab for hCD20 Receptor Binding

Competition of Nb 9079 with Rituximab, Obinituzumab and Ofatumomab for hCD20 receptor binding was analyzed by pre-incubating 5×10⁵ Daudi cells with a 100-fold molar excess of Rituximab, Obinituzumab or Ofatumomab for 1 h at 4° C., prior to incubation with 1 μg/200 of Nb 9079. After washing, Nb binding was detected by incubating the cells with 2 μg Fluorescein IsoThioCyanate (FITC) labeled anti-HisTag Ab (Genscript) for 30 min at 4° C. Mean fluorescence intensity (MFI) was measured using LSR Fortessa Flow Cytometer (BD) and analysed with Flow Jo 7 software (Tree Star Inc., Ashland, Oreg., USA).

### Conjugation of CHX-A″-DTPA to Nbs

Untagged anti-hCD20 Nbs 9077 and 9079, and the nontarget Nb (ctrl Nb) were reconstituted in 0.05M sodium carbonate buffer (pH 8.5) and conjugated with CHX-A″-DTPA for ¹⁷⁷Lu labeling. Briefly, a 10-fold molar excess of CHX-A″-DTPA was added to the Nbs and incubated for 3 h, at room temperature (RT). Adjusting the pH of mixture to 7.0 quenched the reaction. Next, the DTPA-Nb conjugates were purified on Superdex Peptide 10/300 (GE Healthcare) in 0.1M ammonium acetate buffer, pH 7.0. The mean degree of DTPA-conjugation per Nb molecule was determined by ESI-Q-ToF-MS (Waters, Micromass), after which the concentration of the DTPA-Nb conjugates was determined spectrophotometrically at 280 nm by using the corrected molecular weight and extinction coefficient.

### Preparation of ¹⁷⁷Lu-DTPA-Nbs

DTPA-Nb conjugates were radiolabeled with carrier-free ¹⁷⁷Lu, obtained from ITG (Garching, Germany) as a chloride solution with a specific activity of 3000 GBq/mg. In short, the desired activity of ¹⁷⁷Lu (37-350 MBq) was added to a test vial containing 0.2M ammonium acetate buffer (pH 5.0), and incubated with the DTPA-Nb conjugates for 30 min at RT. Next, the mixtures were purified using PBS-equilibrated size-exclusion NAP-5 columns (GE Healthcare), and filtered via a 0.22 μm membrane filter (Millex, Millipore). The radiochemical purities of the final ¹⁷⁷Lu-DTPA-Nbs were evaluated by iTLC-SG and Size-Exclusion Chromatography (SEC) on a Superdex 75 5/15 column (GE Healthcare).

### Preparation of ¹⁷⁷Lu-DTPA-Rituximab

A 100-fold molar excess of CHX-A″-DTPA chelator was conjugated to the free-ε-amino-groups of lysines in Rituximab (MabThera®, Roche Nederland B.V., The Netherlands) in final volume of 3500 μL of 0.05M sodium carbonate buffer (pH 8.5). Reducing the pH to 7.0 quenched the reaction. DTPA-Rituximab was purified by using Superdex 75 10/30 (GE Healthcare) in 0.1 M ammonium acetate buffer, pH 7.0. The DTPA-Rituximab was radiolabeled with ¹⁷⁷Lu and purified as already described for DTPA-Nbs. The radiochemical purity was evaluated by iTLC-SG, and SEC, as described previously.

### Stability of ¹⁷⁷Lu-DTPA-Nbs in Human Serum

50 uL of ¹⁷⁷Lu-DTPA-Nbs was mixed with 1 mL of human serum and incubated at 37° C. during 144 h. Aliquots were taken over time and analyzed on a Superdex 75 5/15 column (GE Healthcare), with 0.01M PBS and 0.3M sodium chloride solution used as mobile phase at a flow rate of 0.3 mL min⁻¹.

### In Vitro Specificity, Affinity and Degree of Internalization of ¹⁷⁷Lu-DTPA-Anti-hCD20 Nbs

Binding specificity, affinity and degree of internalization of ¹⁷⁷Lu-DTPA-Nbs 9077 and 9079 was evaluated on hCD20^{pos} B16 cells. 3.5×10⁴ cells were seeded overnight in 24-well plates to assess specificity of binding to hCD20 receptor. Plates were first placed at 4° C. for 30 min. After removing supernatant and washing cells twice with 1 mL cold PBS, each well was incubated with 20 nM ¹⁷⁷Lu-DTPA-Nbs for 2 h at 4° C., with and without a 100-fold molar excess of unlabeled Rituximab. In parallel, cells were incubated with ¹⁷⁷Lu-DTPA-nontarget Nb (¹⁷⁷Lu-DTPA-ctrl Nb). Next, cells were lysed with 0.5 mL 1M NaOH. The radioactivity present in the lysate was measured using a γ-counter (Cobra Inspector 5003, Canberra Packard, USA) and plotted using Graphpad Prism (version 5.0b). To measure the affinity towards hCD20 receptor, cells were incubated with a serial dilution of ¹⁷⁷Lu-DTPA-Nbs (ranging from 0.1 nM to 250 nM), with and without a 100-fold molar excess of cold Rituximab, and further processed as described above. 1.25×10⁵ cells were adhered overnight in 6-well plates to measure the degree of internalization of ¹⁷⁷Lu-DTPA-Nbs in to hCD20^{pos} B16 cells. The next day, the cells were placed at 4° C. for 1 h. After removing supernatant and washing twice with 1 mL cold PBS, about 20 nM of ¹⁷⁷Lu-DTPA-Nbs was added, with or without a 100-fold molar excess of cold Rituximab. Next, cells were incubated for 2 h at 4° C., after which supernatant was removed and cells were washed twice with 1 mL cold PBS, to obtain the unbound Nb fraction. After adding 2 mL of unsupplemented media in each well, cells were incubated for 0, 1, 2, 4, 6 and 24 h at 37° C. After incubation, supernatant was collected before and after washing cells twice with 2 mL cold PBS in order to collect the dissociated Nb fraction. Next, cells were incubated with 2 mL of 0.05 M Glycine (pH=2.8) for 5 min at 4° C. to obtain the membrane-bound Nb fraction. Again cells were washed twice with 2 mL cold PBS. Finally, each well received 4 mL 0.5 M NaOH, after which cells were incubated for 15 min at 37° C. This suspension was collected as the internalized Nb fraction. The radioactivity present in each fraction was measured using a γ-counter and plotted using Graphpad Prism (version 5.0b).

### Animal Models

For biodistribution experiments, female C57 BL6 mice were subcutaneously inoculated in the right hind limb with 5×10⁵ hCD20^{pos} B16 cells under 2.5% isoflurane anesthesia (Abbott, Ottignier-Louvain-la-Neuve, Belgium). Tumors were allowed to reach a maximal size of 250-350 mm³. Prior to micro-SPECT/CT imaging, mice were anesthetized with a mixture of 18.75 mg/kg⁻¹ ketamine hydrochloride (Ketamine 1000 Ceva®, Ceva, Brussels, Belgium) and 0.5 mg/kg⁻¹ medetomidine hydrochloride (Domitor®, Pfizer, Brussels, Belgium). For therapy experiment, female C57 BL6 mice were subcutaneously inoculated with 3×10⁵ hCD20^{pos} B16 cells. The ethical committee of the Vrije Universiteit Brussel approved all animal study protocols.

### In Vivo Biodistribution and Tumor Targeting of Radiolabeled Anti-hCD20 Nbs

Mice bearing hCD20^{pos} B16 tumors were injected i.v. with ¹⁷⁷Lu-DTPA-Nbs (2.1-10.3 MBq), co-infused with 150 mg/kg Gelofusin. 1 h p.i., micro-SPECT/CT imaging (MILabs VECTor/CT) was performed in mice injected with ¹⁷⁷Lu-DTPA-Nbs. The micro-CT scans was set to 55 kV and 615 μA, resolution of 80 μm. The total body scan was 1 min 48 sec. SPECT images were obtained using rat SPECT collimator (1.5 mm pinholes) in spiral model, 20 positions for whole-body imaging, with 90 sec per position. Images were reconstructed with 0.4 mm voxels with 2 subsets and 7 iterations, without post-reconstruction filter. Analysis of the in vivo biodistribution was done using AMIDE software. The images were generated using the OsiriX Lite software. Mice were euthanized after 1.5 h, followed by the isolation of different organs, tissues and tumors. The present radioactivity in the different samples was measured against a standard of known radioactivity using a γ-counter (Cobra Inspector 5003, Canberra Packard, USA) and expressed as % IA per gram, corrected for decay.

Comparative Ex Vivo Biodistribution of ¹¹⁷Lu-DTPA-Nb 9079 and ¹⁷⁷Lu-DTPA-Rituximab

Mice bearing hCD20^{pos} B16 tumors were injected i.v. with either ¹⁷⁷Lu-DTPA-Nb 9079 in co-injection with 150 mg/kg Gelofusin, or ¹⁷⁷Lu-DTPA-Rituximab (2.9-3.5 MBq; n=3). Mice were euthanized at different time points after injection, followed by the isolation of different organs, tissues and tumors. The present radioactivity in the different samples was measured against a standard of known radioactivity using a γ-counter (Cobra Inspector 5003, Canberra Packard, USA) and expressed as % IA per gram, corrected for decay.

### Dosimetry and Targeted Radionuclide Therapy

Data obtained from the comparative ex vivo biodistribution study were time integrated for the dosimetry calculation of ¹⁷⁷Lu-DTPA-Nb 9079 and ¹⁷⁷Lu-DTPA-Rituximab per gram tissue. Briefly, the integrations between time 0 and 72 h for ¹⁷⁷Lu-DTPA-Nb 9079 and between 0 and 120 h for ¹⁷⁷Lu-DTPA-Rituximab were made using the trapezoid method. In the absorbed dose calculations, S values were obtained from RADAR phantoms (Unit Density Spheres), with S value for a 1 g sphere (0.0233 mGγ/MBq·s) used to calculate all organ doses. For targeted radionuclide therapy, mice bearing hCD20^{pos} B16 tumors (13.2±1.3 mm³) were randomly categorized into 5 groups (n=8). Two groups received 4 i.v. injections, once every two days, of a total accumulative dose of 144±1.8 MBq ¹⁷⁷Lu-DTPA-Nb 9079 or 135±2.74 MBq ¹⁷⁷Lu-DPTA-ctrl Nb. Two other groups received 4 i.v. injections, once every two days, of 200 μg/injection of unlabeled Rituximab or PBS. A final group received a single injection of 7±1.48 MBq ¹⁷⁷Lu-DTPA-Rituximab. The samples containing ¹⁷⁷Lu-DTPA-Nb conjugates were diluted with 150 mg/kg Gelofusin to facilitate clearance from kidneys. Animal weight and tumor volume (caliper) were measured daily. Endpoint criteria were defined as >20% loss of the initial body weight, a tumor volume exceeding 1000 mm³, the presence of necrotic tumors, or finally limb lameness.

### Example 1: Generation and Production of Nbs Against hCD20

Peripheral blood lymphocytes (PBLs) were isolated from the blood of an immunized llama. From the PBLs total RNA was isolated and reverse transcribed into cDNA. From this cDNA, the sequences encoding Nanobodies (the variable domain of the heavy-chain-only antibodies) were amplified by a two-step PCR and cloned in the phagemid vector pMECS. Nanobodies were phage-displayed and used for biopanning on human CD20 transfected CHO cells. Cell ELISA of periplasmic extracts on CHO cells that were either untransfected or transfected with human CD20 revealed several clones that uniquely bound to human CD20. All selected Nb clones were recloned in the bacterial expression vector pHEN6, produced in the E. coli periplasm and purified by osmotic shock, IMAC and size-exclusion chromatography.

### Example 2: Determination of Binding EC50s

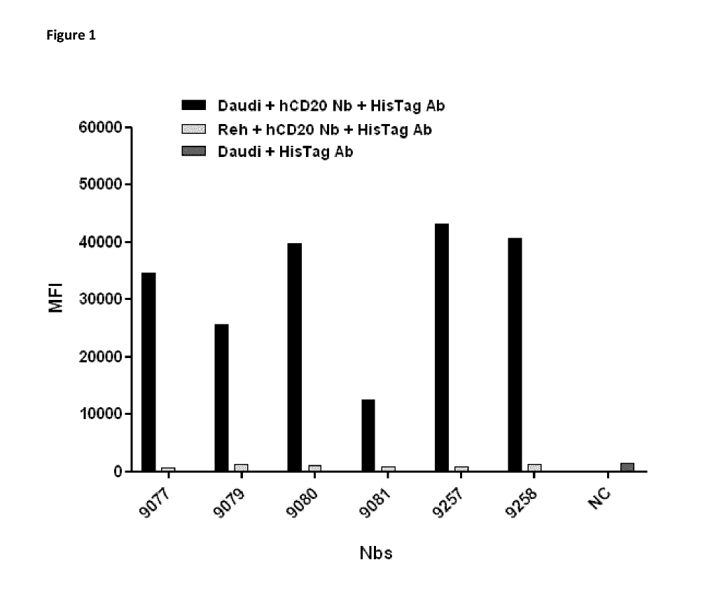

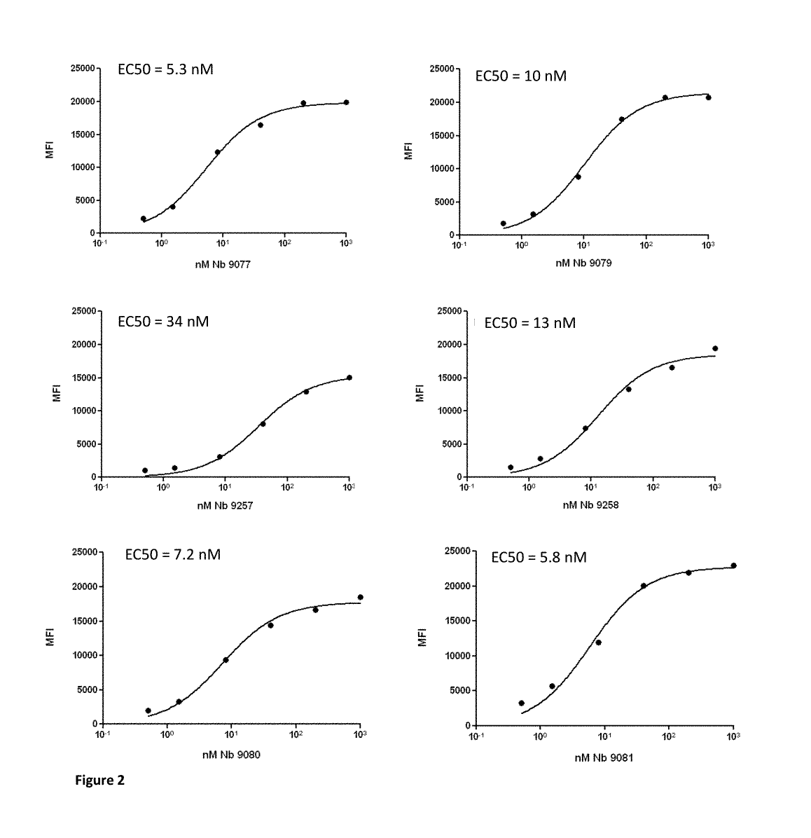

Binding specificity of 6 anti-hCD20 Nbs (Nb 9077, Nb 9079, Nb 9080, Nb 9081, Nb 9257 and Nb 9258) was determined on hCD20+ Daudi and hCD20-Reh cells. MFIs were measured for total binding on both cell lines. All 6 Nbs bound in a specific manner to Daudi cells, but not to Reh cells, as shown in FIG. 1. The binding EC50 of the Nbs to hCD20 was evaluated using flow cytometry on Daudi cells. Each graph in FIG. 2 depicts the MFI for 6 different dilutions of each Nb. Sigmoid curves were obtained for all 6 Nbs, indicating concentration-depending binding of the Nbs to hCD20+ cells. Our results show that all 6 Nbs bind to the hCD20 marker with an EC50 in the nanomolar range.

### Example 3: In Vivo Biodistribution in the Daudi Tumor Xenografted Mouse Model

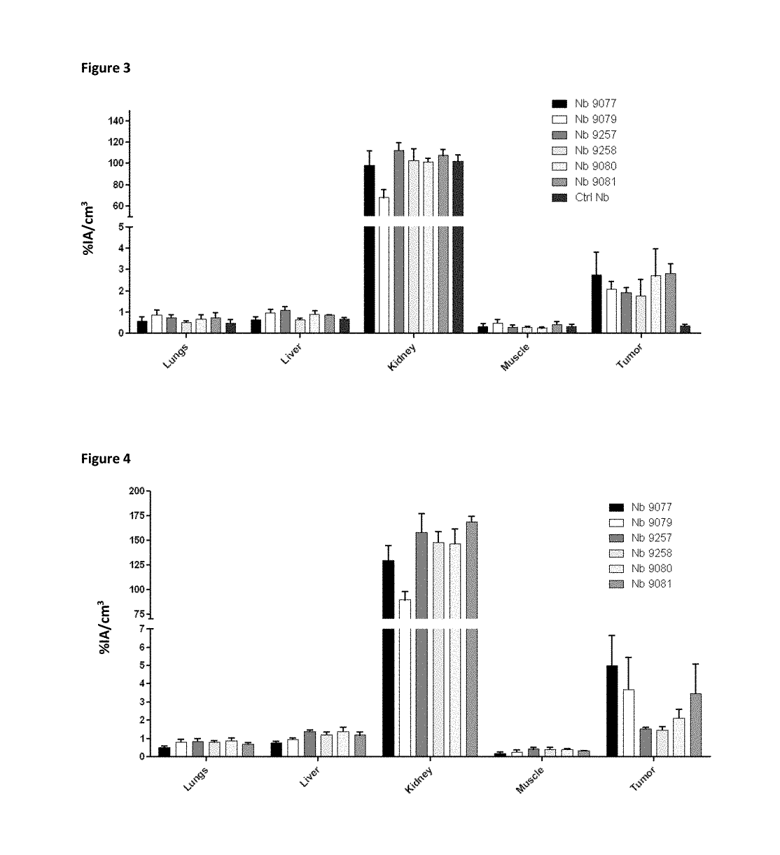

1 h after the intravenous (i.v.) injection of ^{99m}Tc-labeled Nbs in Daudi tumor xenografted mice, SPECT/micro-CT images were taken and uptake values in different organs and tissues were calculated using AMIDE software. Results obtained for lung, liver, kidney, muscle, and tumor tissue are shown in FIG. 3. These results demonstrate the in vivo tumor-targeting capacity of all 6 hCD20-specific Nbs. Notably, all 6 anti-hCD20^{99m}Tc-Nbs demonstrate higher tumor uptake than the non-target control ^{99m}Tc-Nb (Ctrl Nb), suggesting specific uptake in the tumor. Only little accumulation was observed in lungs, muscle, and liver for all Nbs. Higher uptake of radioactivity was observed in kidneys for all Nbs. However, surprisingly, ^{99m}Tc-Nb 9079 showed lower kidney accumulation compared to the five other ^{99m}Tc-Nbs.

### Example 4: In Vivo Biodistribution in the B16 Tumor Mouse Model

1 h after the i.v. injection of ^{99m}Tc-labeled Nbs in hCD20^{pos} B16 tumor mice, SPECT/micro-CT images were taken and uptake values in different organs and tissues were calculated using AMIDE software. Results obtained for lung, liver, kidney, muscle, and tumor tissue are shown in FIG. 4. The results obtained are in agreement with those obtained in the Daudi tumor mouse model (Example 3). Surprisingly, ^{99m}Tc-Nb 9079 showed lower kidney accumulation compared to the five other ^{99m}Tc-Nbs, while other organs and tissues showed similar uptake values for all 6 Nbs.

### Example 5: Ex Vivo Biodistribution in the Daudi Tumor Xenografted Mouse Model

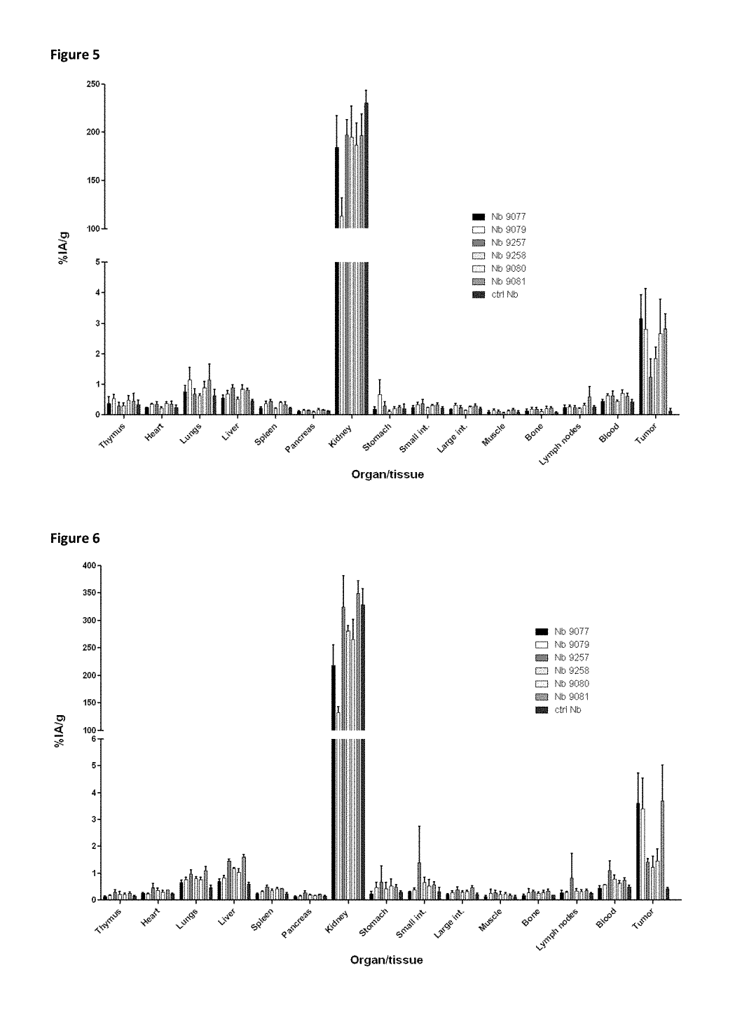

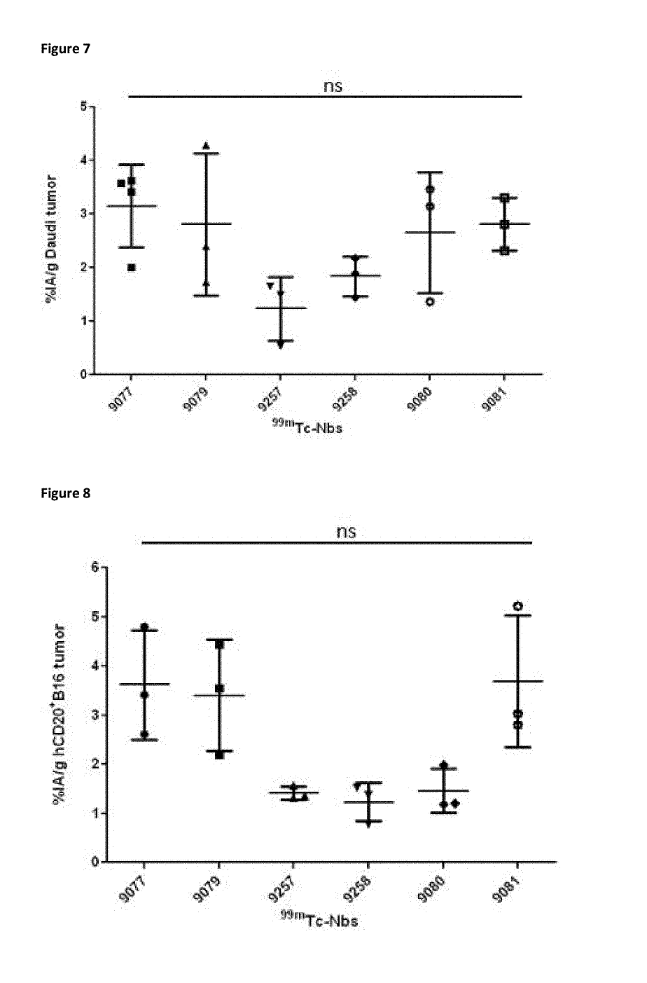

1.5 h after the i.v. injection of ^{99m}Tc-labeled Nbs in Daudi tumor xenografted animals, the animals were sacrificed and dissected. Tissues and organs of interest were isolated, weighed and measured for radioactivity. Uptake values in different organs and tissues were calculated. Results obtained for lung, liver, kidney, muscle, and tumor are shown in FIG. 5. These results confirm the in vivo tumor-targeting capacity of all 6 Nbs. All 6 anti-hCD20^{99m}Tc-Nbs demonstrate higher tumor uptake than the non-target control ^{99m}Tc-Nb, suggesting specific uptake in the tumor. No significant difference in tumor uptake was observed between the 6 Nbs (FIG. 7). Only little non-specific accumulation was observed in lungs, muscle, and liver for all Nbs. Higher uptake of radioactivity was observed in kidneys for all Nbs. Surprisingly, ^{99m}Tc-Nb 9079 showed significant lower kidney accumulation compared to the five other ^{99m}Tc-Nbs (FIG. 5).

### Example 6: Ex Vivo Biodistribution in the hCD20^{pos} B16 Tumor Mouse Model

1.5 h after the i.v. injection of ^{99m}Tc-labeled Nbs in Daudi tumor xenografted animals, the animals were sacrificed and dissected. Tissues and organs of interest were isolated, weighed and measured for radioactivity. Uptake values in different organs and tissues were calculated. Results obtained for the indicated organs/tissues are shown in FIG. 6. The results obtained here are in close agreement with those obtained in the Daudi tumor mouse model. Surprisingly, ^{99m}Tc-Nb 9079 showed significant lower kidney accumulation compared to the 5 other ^{99m}Tc-Nbs (FIG. 6), while no significant difference in tumor uptake was observed (FIG. 8).

### Example 7: Kidney Accumulation of Nb 9079

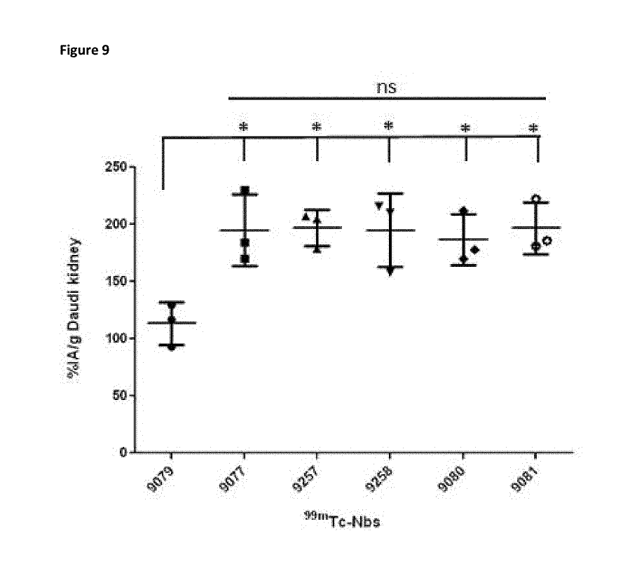

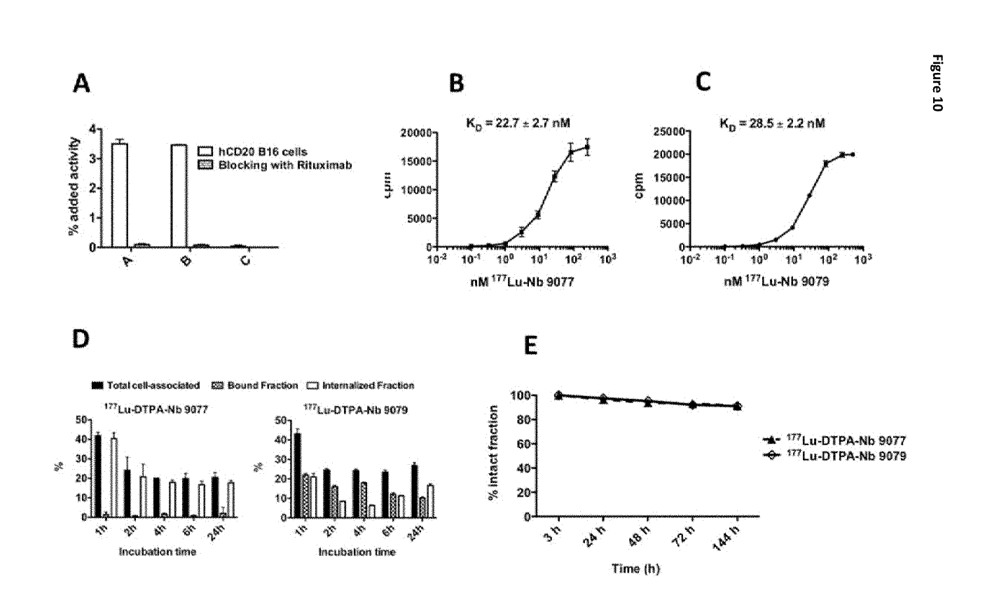

As shown in FIG. 9, the kidney retention of Nb 9079 was surprisingly lower compared to the other evaluated anti-hCD20 Nbs, although its general biodistribution is not different compared to that of the five additional Nbs.

### Example 8: Development and In Vitro Characterization of ¹⁷⁷Lu-DTPA-Nb 9077 and 9079

So far, the above described Nb 9077 and 9079 were labeled with Technetium (^{99m}TC). This radioisotope is most widely used in medicine because it has almost ideal characteristics for nuclear medicine scans. These are: it has a half-life of six hours which is long enough to examine metabolic processes yet short enough to minimize the radiation dose to the patient. Technetium-99m decays by a process called “isomeric”; which emits gamma rays and low energy electrons. Since there is no high energy beta emission, the radiation dose to the patient is low. Although ^{99m}Tc-Nb are thus well suited as diagnostic radiopharmaceuticals, the ^{99m}Tc label because of its low radiation intensity cannot be used for therapeutic purposes. Indeed, for some medical conditions, it is useful to destroy or weaken malfunctioning cells using radiation. In most cases, it is beta radiation which causes the destruction of the damaged cells. An ideal therapeutic radioisotope is a beta emitter with just enough gamma to enable imaging, eg lutetium-177. The radioisotope that generates a therapeutic radiation dose can be localised in the required organ in the same way it is used for diagnosis, i.e. through a radioactive element following its usual biological path, or through the element being attached to a suitable biological compound. To evaluate the therapeutic efficacy of radiolabeled anti-hCD20 Nbs, we labelled Nb 9077, Nb 9079 and a control Nb that does not bind CD20 with ¹⁷⁷Lu. The specificity of binding was measured on hCD20^{pos} B16 cells. ¹⁷⁷Lu-DTPA-anti-hCD20 Nbs 9077 and 9079 were incubated at 20 nM with cells for 2 h at 4° C. Specific binding of the ¹⁷⁷Lu-DTPA-anti-hCD20 Nbs were presented as binding of ¹⁷⁷Lu-DTPA-anti-hCD20 Nbs (total) versus binding in the presence of a 100-fold molar excess of Rituximab (blocked), and versus binding of ¹⁷⁷Lu-DTPA-nontarget Nb (¹⁷⁷Lu-DTPA-ctrl Nb) (FIG. 10 A). Binding affinity towards hCD20 receptor was calculated by incubating serial dilutions of ¹⁷⁷Lu-DTPA-anti-hCD20 Nbs with hCD20^{pos} B16 cells 2 h at 4° C. The concentration-response curves for both ¹⁷⁷Lu-DTPA-anti-hCD20 Nbs are presented in FIGS. 10 B and C. K_{D} values were obtained, with 22.7±2.7 nM and 28.5±2.2 nM for ¹⁷⁷Lu-DTPA-anti-hCD20 Nbs 9077 and 9079, respectively (FIGS. 10 B and C). To investigate potential internalization upon hCD20 receptor binding, cells were incubated with 20 nM of the ¹⁷⁷Lu-DTPA-anti-hCD20 Nbs 9077 and 9079. The degree of internalization was analyzed at different time points, with or without 100-fold molar excess of Rituximab. After 1 h, about 40.4±3% and 21±1.8% of initial bound activity was internalized for ¹⁷⁷Lu-DTPA-anti-hCD20 Nbs 9077 and 9079, respectively. After 24 h, the internalized fractions decreased to 17.69±1.2% and 16.6±0.9% for ¹⁷⁷Lu-DTPA-Nb 9077 and 9079, respectively (FIG. 10 D). In addition, when we analyzed the stability of ¹⁷⁷Lu-DTPA-Nb 9077 and 9079 in human serum, both anti-hCD20¹⁷⁷Lu-labeled Nbs showed to be stable in human serum at 37° C. for multiple days, with still more than 91% intact complexes after 144 h (FIG. 10 E).

### Example 9: In Vivo Distribution of 177Lu-DTPA-Nb 9077 and 9079

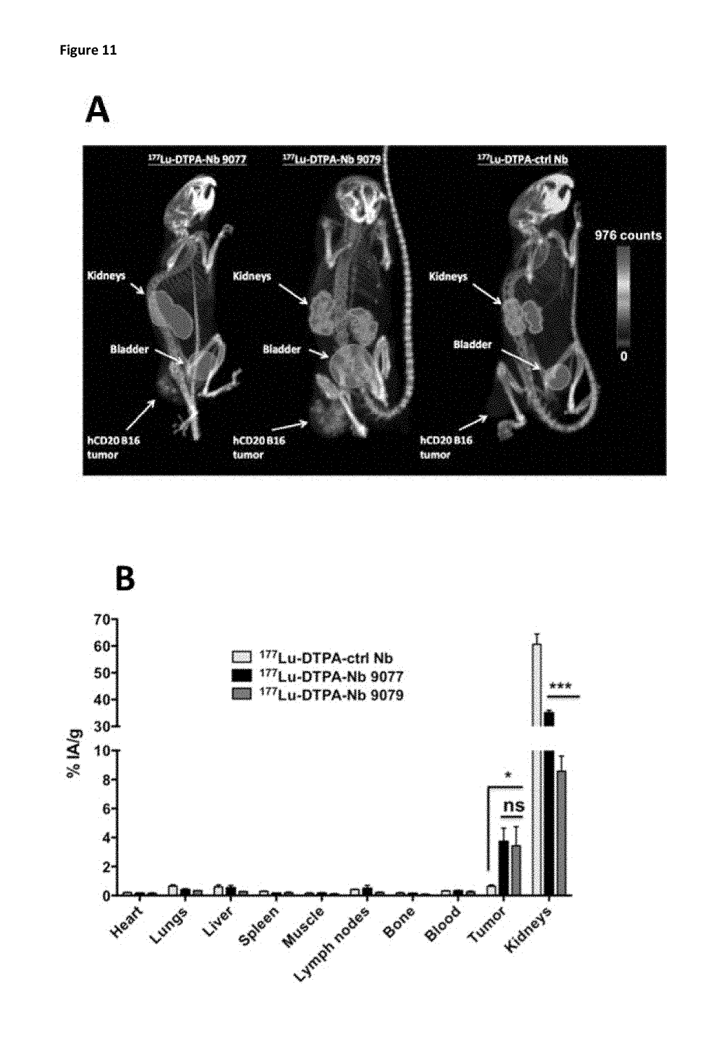

Biodistribution and tumor-targeting of ¹⁷⁷Lu-DTPA-anti-hCD20-Nbs 9077 and 9079 was assessed in mice bearing hCD20^{pos} B16 tumors. Micro-SPECT/CT images were generated 1 h after i.v. injection, followed by dissections after 1.5 h. In vivo images showed specific tumor targeting for both ¹⁷⁷Lu-DTPA-anti-hCD20 Nbs, with a low background signal, except kidneys and bladder (FIG. 11 A). The ex vivo biodistribution data generated via dissections (FIG. 11 B and Table 1) revealed similar tumor targeting for both ¹⁷⁷Lu-DTPA-anti-hCD20 Nbs, with 3.7±0.9% and 3.4±1.3% IA/g for Nb 9077 and Nb 9079, respectively. Analysis of ¹⁷⁷Lu-DTPA-nontarget Nb (¹⁷⁷Lu-DTPA-ctrl Nb) noted a tumor uptake of only 0.64±0.09% IA/g, significant lower than ¹⁷⁷Lu-DTPA-anti-hCD20 Nbs (p<0.012), confirming the specific targeting of both anti-hCD20 Nbs (Table 1). The uptake values in the additional organs and tissues were below 0.5% IA/g, except in kidneys. A significant difference in kidney uptake was observed between ¹⁷⁷Lu-DTPA-anti-hCD20 Nb 9079 (8.6±1.1% IA/g) compared to ¹⁷⁷Lu-DTPA-anti-hCD20 Nb 9077 (35±0.98% IA/g) (Table 1). These results confirm the earlier described results from the ^{99m}Tc labeled anti-hCD20 Nb 9077 and 9079, i.e. the anti-hCD20 Nb 9079 shows a surprisingly lower uptake in the kidney. This is an extremely important achievement since lower renal retention of radioisotope labeled compounds significantly increases the medical potentials.

TABLE 1
Ex vivo biodistribution of ¹⁷⁷Lu-DTPA-Nb 9077, 9079 and nontarged_Nb (¹⁷⁷Lu-DTPA-ctrl
Nb) at 1.5 h p.i., co-infused with 150 mg/kg Gelofusin, in hCD20^{pos} B16 tumor mouse model.
Results are presented as mean % IA/g ± SD (n = 3 per Nb).

| | ¹⁷⁷Lu-DTPA-Nb 9077 | ¹⁷⁷Lu-DTPA-Nb 9079 | ¹⁷⁷Lu-DTPA-ctrl Nb |
|---|---|---|---|
| Thymus | 0.10 ± 0.02 | 0.11 ± 0.04 | 0.14 ± 0.01 |
| Heart | 0.15 ± 0.04 | 0.16 ± 0.04 | 0.21 ± 0.02 |
| Lungs | 0.40 ± 0.08 | 0.34 ± 0.01 | 0.65 ± 0.10 |
| Liver | 0.53 ± 0.18 | 0.28 ± 0.00 | 0.60 ± 0.13 |
| Spleen | 0.16 ± 0.01 | 0.20 ± 0.05 | 0.29 ± 0.02 |
| Pancreas | 0.15 ± 0.03 | 0.13 ± 0.02 | 0.23 ± 0.08 |
| Kidneys | 35.02 ± 0.98 | 8.58 ± 1.05*** | 60.64 ± 3.85 |
| Stomach | 0.30 ± 0.05 | 0.4 ± 0.24 | 0.42 ± 0.15 |
| S Intestine | 0.38 ± 0.22 | 0.35 ± 0.06 | 0.41 ± 0.34 |
| L Intestine | 0.21 ± 0.04 | 0.26 ± 0.10 | 0.47 ± 0.14 |
| Muscle | 0.16 ± 0.04 | 0.11 ± 0.03 | 0.16 ± 0.04 |
| Bone | 0.14 ± 0.04 | 0.09 ± 0.01 | 0.18 ± 0.04 |
| L Nodes | 0.48 ± 0.23 | 0.22 ± 0.02 | 0.42 ± 0.02 |
| Blood | 0.31 ± 0.05 | 0.26 ± 0.05 | 0.32 ± 0.02 |
| Tumor | 3.72 ± 0.93 | 3.44 ± 1.31 | 0.64 ± 0.09 |
| T/M | 25.7 ± 13.5 | 32.9 ± 15.6 | 4.2 ± 0.7 |
| T/B | 11.8 ± 1.1 | 13.3 ± 4.6 | 1.9 ± 0.2 |

T/M: Tumor-to-Muscle ratio; T/B: Tumor-to-Blood ratio. ¹⁷⁷Lu-DTPA-Nb 9079 showed significant lower kidney accumulation than ¹⁷⁷Lu-DTPA-Nb 9077 (p < 0.0001)

Next, we compared the ex vivo biodistribution of ¹⁷⁷Lu-DTPA-Nb 9079 with ¹⁷⁷Lu-DTPA-Rituximab. ¹⁷⁷Lu-DTPA-Nb 9079 showed the highest tumor uptake values after 1.5 h (3.4±1.3% IA/g), which decreased to 0.86±0.13% IA/g after 24 h and to 0.35±0.04% IA/g after 72 h. Kidney accumulation was also the highest at early time points and decreased from 8.56±1.05% IA/g at 1.5 h p.i. to 1.47±0.46% IA/g at 24 h p.i. and to 0.22±0.05% IA/g after 72 h p.i. Radioactivity accumulation in the other non-target organs and tissues was below 0.5% IA/g at 1.5 h p.i. and decreased over time. In contrast, the kinetics of ¹⁷⁷Lu-DTPA-Rituximab are opposite to those obtained for ¹⁷⁷Lu-DTPA-Nb 9079, with lower tumor uptake at early time points, which than increased from 10.65±1.86% IA/g at 1.5 h p.i. to 27.36±5.46% IA/g at 120 h p.i. Blood values for ¹⁷⁷Lu-DTPA-Rituximab were higher than tumor values at all-time points with 98.05±13.29% IA/g at 1.5 h p.i. and 35.05±5.18% IA/g at 120 h p.i. At all-time points, the radioactivity accumulation of ¹⁷⁷Lu-DTPA-Rituximab in the other non-target organs and tissues was very high compared to that of ¹⁷⁷Lu-DTPA-Nb 9079.

TABLE 2
Ex vivo biodistribution of ¹⁷⁷Lu-DTPA-Nb 9079 co-infused with 150 mg/kg Gelofusin and ¹⁷⁷Lu-DTPA-
Rituximab, in hCD20^{pos} B16 tumor mouse model at different time-points after i.v. administration
(n = 3 per time point). Results are presented as mean % IA/g ± SD. T/K: Tumor-to-
Kidneys ratio; T/M: Tumor-to-Muscle ratio; T/B: Tumor-to-Blood ratio; NA: Not Analyzed

| | | | | | |
|---|---|---|---|---|---|
| 1.5 h | 6 h | 24 h | 48 h | 72 h | 120 h |

177Lu-DTPA-Nb 9079

| | | | | | | |
|---|---|---|---|---|---|---|
| Thymus | 0.11 ± 0.04 | 0.04 ± 0.00 | 0.05 ± 0.02 | 0.02 ± 0.01 | 0.06 ± 0.09 | NA |
| Heart | 0.16 ± 0.04 | 0.07 ± 0.00 | 0.02 ± 0.00 | 0.01 ± 0.00 | 0.01 ± 0.00 | NA |
| Lungs | 0.34 ± 0.01 | 0.14 ± 0.00 | 0.03 ± 0.01 | 0.02 ± 0.01 | 0.01 ± 0.00 | NA |
| Liver | 0.28 ± 0.00 | 0.22 ± 0.06 | 0.09 ± 0.01 | 0.05 ± 0.01 | 0.04 ± 0.00 | NA |
| Spleen | 0.20 ± 0.05 | 0.09 ± 0.01 | 0.10 ± 0.01 | 0.04 ± 0.01 | 0.03 ± 0.01 | NA |
| Pancreas | 0.13 ± 0.02 | 0.08 ± 0.02 | 0.02 ± 0.00 | 0.01 ± 0.00 | 0.01 ± 0.00 | NA |
| Kidneys | 8.58 ± 1.05 | 6.33 ± 1.53 | 1.47 ± 0.46 | 0.38 ± 0.08 | 0.22 ± 0.05 | NA |
| Stomach | 0.4 ± 0.24 | 0.30 ± 0.29 | 0.08 ± 0.10 | 0.01 ± 0.00 | 0.01 ± 0.00 | NA |
| S Intestine | 0.35 ± 0.06 | 0.49 ± 0.11 | 0.07 ± 0.02 | 0.01 ± 0.00 | 0.01 ± 0.01 | NA |
| L Intestine | 0.26 ± 0.10 | 0.51 ± 0.15 | 0.10 ± 0.10 | 0.02 ± 0.01 | 0.04 ± 0.01 | NA |
| Muscle | 0.11 ± 0.03 | 0.05 ± 0.03 | 0.01 ± 0.01 | 0.00 ± 0.00 | 0.00 ± 0.00 | NA |
| Bone | 0.09 ± 0.01 | 0.07 ± 0.00 | 0.03 ± 0.01 | 0.03 ± 0.00 | 0.03 ± 0.00 | NA |
| L Nodes | 0.22 ± 0.02 | 0.11 ± 0.02 | 0.06 ± 0.01 | 0.02 ± 0.01 | 0.05 ± 0.02 | NA |
| Blood | 0.26 ± 0.05 | 0.06 ± 0.04 | 0.01 ± 0.00 | 0.00 ± 0.00 | 0.00 ± 0.00 | NA |
| Tumor | 3.44 ± 1.31 | 1.63 ± 0.07 | 0.86 ± 0.13 | 0.54 ± 0.07 | 0.35 ± 0.04 | NA |
| T/K | 0.41 ± 0.17 | 0.26 ± 0.04 | 0.63 ± 0.25 | 2.48 ± 0.39 | 1.63 ± 0.5 | NA |
| T/M | 32.97 ± 15.57 | 51.51 ± 26.37 | 104 ± 73.27 | 163.28 ± 11.76 | 97.81 ± 27.74 | NA |
| T/B | 13.35 ± 4.6 | 27.78 ± 16.32 | 159.7 ± 72.3 | 199.75 ± 93.91 | 134.83 ± 32.39 | NA |

177Lu-DTPA-Rituximab

| | | | | | | |
|---|---|---|---|---|---|---|
| Thymus | 16.3 ± 4.8 | 13.6 ± 1.51 | 6.49 ± 0.32 | 7.90 ± 0.43 | 8.11 ± 0.81 | 8.41 ± 0.86 |
| Heart | 22.9 ± 0.99 | 22.4 ± 7.37 | 11.5 ± 3.89 | 10.6 ± 0.99 | 6.10 ± 2.15 | 9.64 ± 1.84 |
| Lungs | 29.1 ± 5.39 | 21.1 ± 1.82 | 12.3 ± 0.89 | 10.7 ± 1.08 | 16.2 ± 2.91 | 13.9 ± 1.70 |
| Liver | 25.4 ± 2.17 | 20.8 ± 0.51 | 12.5 ± 0.24 | 12.1 ± 1.11 | 11.2 ± 1.54 | 12.4 ± 0.87 |
| Spleen | 22.8 ± 3.09 | 20.2 ± 0.33 | 15.7 ± 1.88 | 17.9 ± 2.69 | 15.7 ± 2.01 | 12.4 ± 7.20 |
| Pancreas | 9.6 ± 3.24 | 6.18 ± 1.20 | 4.7 ± 0.54 | 4.15 ± 0.69 | 4.79 ± 0.36 | 3.7 ± 0.60 |
| Kidneys | 28.3 ± 3.71 | 18.9 ± 1.94 | 12.7 ± 0.28 | 10.8 ± 1.24 | 11.9 ± 0.85 | 14.5 ± 0.33 |
| Stomach | 5.10 ± 1.39 | 3.70 ± 0.90 | 2.81 ± 0.48 | 2.78 ± 0.88 | 2.27 ± 0.54 | 2.10 ± 0.38 |
| S Intestine | 7.41 ± 1.3 | 5.83 ± 0.90 | 6.12 ± 4.79 | 6.81 ± 5.81 | 2.61 ± 0.53 | 2.77 ± 0.42 |
| L Intestine | 2.87 ± 2.18 | 5.74 ± 1.01 | 3.49 ± 0.99 | 5.71 ± 4.87 | 2.17 ± 0.22 | 2.60 ± 0.46 |
| Muscle | 3.00 ± 1.24 | 3.51 ± 0.38 | 2.94 ± 0.20 | 2.88 ± 0.38 | 2.61 ± 0.36 | 3.95 ± 0.61 |
| Bone | 9.13 ± 3.23 | 17.2 ± 1.37 | 15.2 ± 2.01 | 33.5 ± 10.25 | 48.8 ± 14.90 | 77.4 ± 14.93 |
| L Nodes | 4.41 ± 4.58 | 12.4 ± 0.10 | 10.7 ± 2.56 | 11.6 ± 1.92 | 11.5 ± 6.27 | 16.4 ± 1.74 |
| Blood | 98.1 ± 13.3 | 71.0 ± 1.49 | 37.2 ± 2.57 | 34.3 ± 3.52 | 29.7 ± 6.05 | 35.1 ± 5.18 |
| Tumor | 10.7 ± 1.86 | 19.9 ± 5.27 | 23.5 ± 3.37 | 21.9 ± 1.81 | 17.8 ± 2.95 | 27.4 ± 5.40 |
| T/K | 0.38 ± 0.07 | 1.01 ± 0.13 | 1.85 ± 0.31 | 2.07 ± 0.39 | 1.48 ± 0.15 | 1.88 ± 0.33 |
| T/M | 4.3 ± 2.86 | 5.68 ± 1.51 | 7.96 ± 0.62 | 7.72 ± 1.27 | 6.83 ± 0.68 | 7.13 ± 2.37 |
| T/B | 0.11 ± 0.02 | 0.29 ± 0.05 | 0.63 ± 0.09 | 0.65 ± 0.09 | 0.6 ± 0.02 | 0.78 ± 0.13 |

### Example 10. Dosimetry and Therapeutic Efficacy of ¹⁷⁷Lu-DTPA-Nb 9079

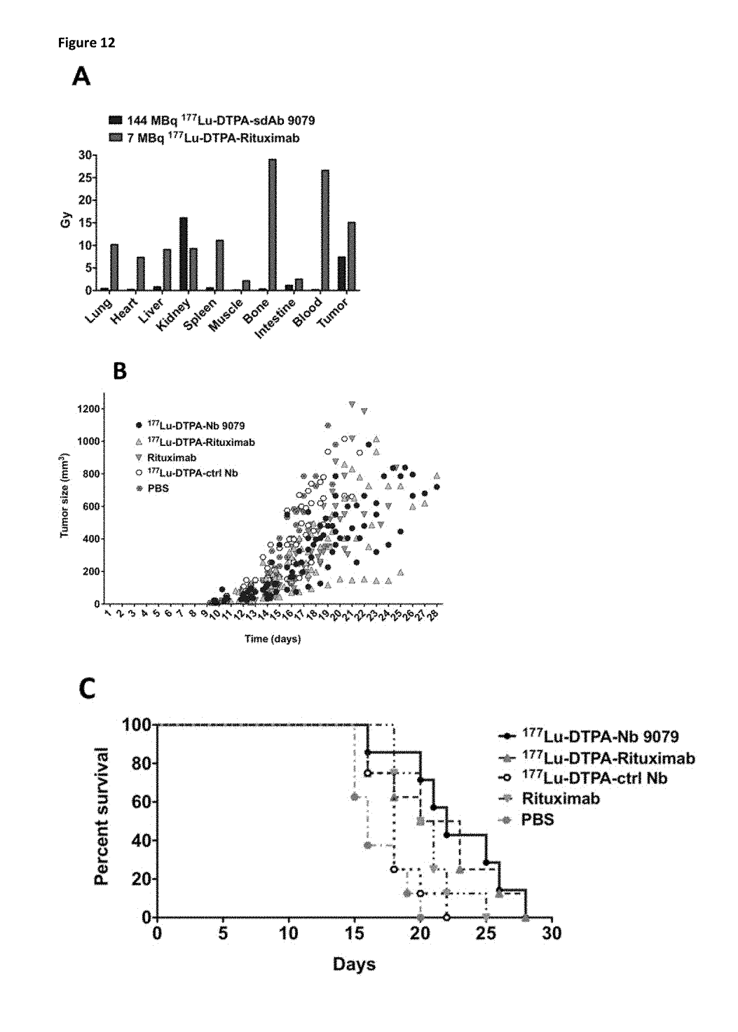

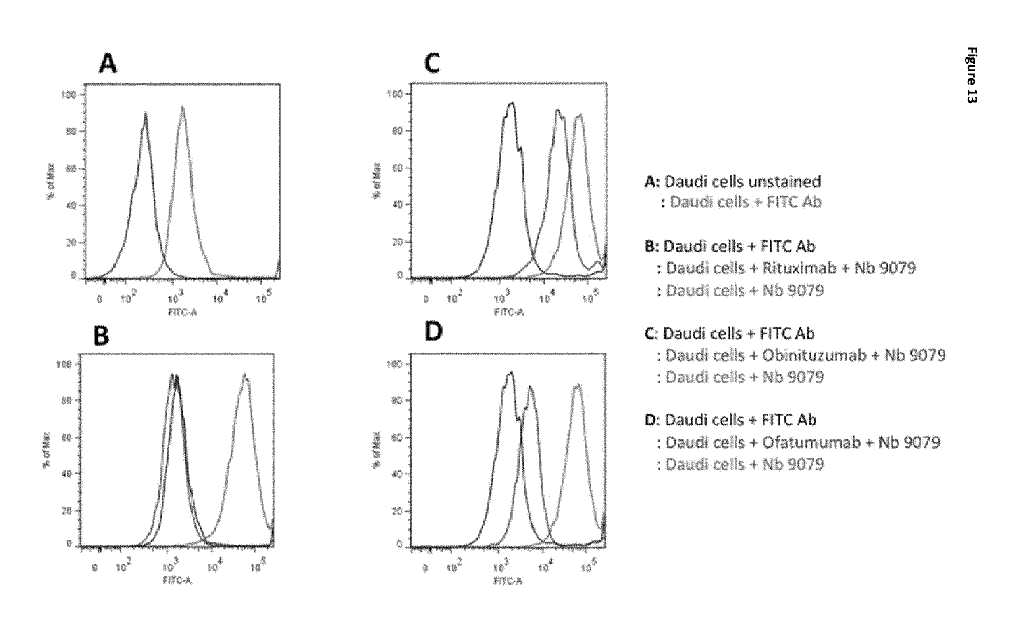

Compared to Nb 9077 as well as to Rituximab, Nb 9079 showed a surprisingly low presence in the kidneys (Tables 1 and 2). Moreover, compared to Rituximab, all distribution values (including presence in tumor) for Nb 9079 were lower. However, the Tumor/Blood values for Nb 9079 were significantly higher compared to those for Rituximab (Table 2). Organ-absorbed doses from 144 MBq of ¹⁷⁷Lu-DTPA-Nb 9079 and 7 MBq ¹⁷⁷Lu-DTPA-Rituximab are depicted in FIG. 12 A. The absorbed dose from ¹⁷⁷Lu-DTPA-Nb 9079 to tumor was 7.4 Gy, while kidneys received a dose of 16.08 Gy. Doses delivered to other healthy organs and tissues were low. 7 MBq of ¹⁷⁷Lu-DTPA-Rituximab led to an absorbed dose of 15 Gy to tumor, but in parallel to doses of 26.6, 29 and 11.1 Gy to blood, bone and spleen, respectively. Absorbed doses to additional organs and tissues were also much higher compared to ¹⁷⁷Lu-DTPA-9079. A lower absolute presence of Nb 9079 in tumor could result in a reduced radiotherapeutic efficacy. To assess therapeutic efficacy, four groups of mice (n=8 per group) received four i.v. injection of ¹⁷⁷Lu-DTPA-Nb 9079 (cumulative radioactive dose of 141±1.8 MBq), or ¹⁷⁷Lu-DTPA-ctrl Nb (cumulative radioactive dose of 135±2.74 MBq), Rituximab (200 μg/injection) or PBS. One group of mice received one i.v. injection of 7 MBq ¹⁷⁷Lu-DTPA-Rituximab. Tumor volumes were quantified using caliper measurements (mm³), in function of time (days; FIG. 12 B). The resulting Kaplan-Meier survival curves are presented in FIG. 12 C. A significant difference in median survival was observed between mice treated with ¹⁷⁷Lu-DTPA-Nb 9079 and ¹⁷⁷Lu-DTPA-ctrl Nb (p<0.02) or PBS (p<0.001). No significant difference in median survival was observed between mice treated with ¹⁷⁷Lu-DTPA-Nb 9079, ¹⁷⁷Lu-DTPA-Rituximab, or Rituximab.

To summarize, in this application, Applicants describe a radiolabeled anti-hCD20 Nb (i.e. ¹⁷⁷Lu-DTPA-Nb 9079) that shows overall significantly lower biodistribution levels compared to currently market-approved ¹⁷⁷Lu-DTPA-Rituximab, while having the same therapeutic efficacy. Moreover, the anti-hCD20 Nb 9079 shows a surprisingly lower renal retention compared to other generated anti-hCD20 Nbs.

### Example 11. In Vitro Competition of Nb9079 with Anti-CD20 mAbs

Rituximab, the market-approved anti-CD20 Ab, is used as first line treatment for cancer in combination with chemotherapy. However, as Rituximab shows a long retention in the patient after administration, a radiolabeled version of Rituximab would have too much detrimental effects on healthy B-cells. Therefore, Zevalin (trade name for ⁹⁰Y-tiuxetan Ibritumomab) was developed. In contrast to Rituximab (a chimeric anti-CD20 monoclonal antibody), Zevalin is a mouse anti-CD20 antibody labeled with radioactivity. First, patients are treated with high doses of Rituximab to block most of the non-target B-cell CD20 epitopes, where after Zevalin is administrated. To test, whether Nb9079 could be used as alternative for Zevalin, an in vitro competition experiment was set up for Rituximab and Nb9079. Daudi cells were pre-incubated with a 100-fold molar excess of Rituximab for 1 h at 4° C., prior to incubation with 1 μg/200 μL of Nb 9079. After washing, Nb binding was detected by incubating the cells with 2 μg Fluorescein IsoThioCyanate (FITC) labelled anti-HisTag Ab for 30 min at 4° C. Mean fluorescence intensity (MFI) measurements using flow cytometry suggests the competition of Nb 9079 with Rituximab (FIG. 13 B). A possible explanation is that Rituximab binds the same epitope as Nb 9079 or that Rituximab blocks the binding of Nb 9079 on hCD20^{pos} B16 cells by steric hindrance. However, when in vitro competition of Nb 9079 with two other anti-CD20 Abs (i.e. Obinutuzumab and Ofatumumab) was tested, partial competition of Nb 9079 with Obinutuzumab was observed (FIG. 13 C, D).

The competition with Rituximab has the advantage that Rituximab could be used to block the CD20 receptor on the healthy non-target B-cells. Indeed, patients treated with Zevalin® first receive relatively high doses of Rituximab prior to Zevalin® administration. In this way, Rituximab blocks the CD20 receptor on the normal B-cells, resulting in decreased radiation of healthy non-target organs and improved tumor targeting of Zevalin®.

As FIG. 13B shows, radiolabeled Nb9079 can thus be used as alternative for Zevalin. However and importantly, Nb9079 has two major therapeutic advantages: 1) Nb9079 has a significantly lower toxicity profile compared to that of Zevalin and administration of Nb9079 will thus lead to less side-effects and 2) radiolabeled Nb9079 can be used more than once without losing efficacy. Indeed, in order to have a fast blood clearance of radiolabeled-mAb, Zevalin® uses mouse mAb Ibritumomab as a targeting vehicle. Murine mAbs are known to interact weaker with human Fc-receptor, resulting in a faster clearance. However, human anti-murine antibodies (HAMAs) are observed in some patients treated with only one therapeutic dose of Zevalin®. In case of repeated injection of Zevalin®, HAMA response result in an altered pharmacokinetics of the therapeutic radiolabeled-mAb.

### REFERENCES

De Vos, J., Devoogdt, N., Lahoutte, T., and Muyldermans, S. (2013). Camelid single-domain antibody-fragment engineering for (pre)clinical in vivo molecular imaging applications: adjusting the bullet to its target. Expert Opin Biol Ther 13, 1149-1160.
D'Huyvetter M., Vincke C, Xavier C, Aerts A, Impens N, Baatout S, De Raeve H, Muyldermans S, Caveliers V, Devoogdt N, Lahoutte T (2014) Targeted Radionuclide Therapy with a 177Lu-labeled Anti-HER2 Na nobody. Theranostics 4, 708-720.
D'Huyvetter M, Xavier C., Caveliers V., Lahoutte T., Muyldermans S., Devoogdt N. (2014). Radiolabeled nanobodies as theranostic tools in targeted radionuclide therapy of cancer. Expert Opin Drug Deliv 18, 1-16.
Emmanouilides, C. (2007). Radioimmunotherapy for non-hodgkin lymphoma: historical perspective and current status. J Clin Exp Hematop. 47, 43-60.
Ersahin, D., Doddamane, I., and Cheng, D. (2011). Targeted radionuclide therapy. Cancers 3, 3838-3855.
Tomblyn, M. (2012). Radioimmunotherapy for B-cell non-hodgkin lymphomas. Cancer Control 19, 196-203.
Wesolowski, J., Alzogaray, V., Reyelt, J., Unger, M., Juarez, K., Urrutia, M., Cauerhff, A., Danguah, W., Rissiek, B., Scheuplein, F., Schwarz, N., Adriouch, S., Boyer, O., Seman, M., Licea, A., Serreze, D. V., Goldbaum, F. A., Haag, F., Koch-Nolte, F. (2009). Single domain antibodies: promising experimental and therapeutic tools in infection and immunity. Med Microbiol Immunol 198, 157-174.

1. A CD20 binding agent, the binding agent comprising:
three complementarity determining regions (CDR1, CDR2, and CDR3),
wherein CDR1 comprises an amino acid sequence with at least 90% sequence identity with the amino acid sequence of SEQ ID No. 1;
wherein CDR2 comprises an amino acid sequence with at least 90% sequence identity with the amino acid sequence of SEQ ID No. 2;
wherein CDR3 comprises an amino acid sequence with at least 90% sequence identity with the amino acid sequence of SEQ ID No. 3; and
wherein the CD20 binding agent is coupled to a radionuclide.

2. The CD20 binding agent of claim 1, wherein the CD20 binding agent comprises a full length antibody or fragment thereof.

3. The CD20 binding agent of claim 1, wherein the CD20 binding agent comprises a single domain antibody.

4.-9. (canceled)

10. A nucleic acid comprising:
a nucleic acid sequence encoding an amino acid sequence comprising at least three complementarity determining regions (CDR1, CDR2, and CDR3),
wherein CDR1 comprises an amino acid sequence with at least 90% sequence identity with the amino acid sequence of SEQ ID No. 1;
wherein CDR2 comprises an amino acid sequence with at least 90% sequence identity with the amino acid sequence of SEQ ID No. 2; and
wherein CDR3 comprises an amino acid sequence with at least 90% sequence identity with the amino acid sequence of SEQ ID No. 3.

11. A vector comprising the nucleic acid of claim 10.

12. A host cell comprising the nucleic acid of claim 10.

13. A pharmaceutical composition comprising:
the CD20 binding agent of claim 1; and
a pharmaceutically acceptable carrier.

14. A method of in vivo medical imaging, the method comprising:
administering to a subject an effective amount of the CD20 binding agent of claim 1; and
detecting the CD20 binding agent in body areas of said subject.

15. (canceled)

16. A method for treating a disease or disorder involving cells expressing CD20, the method comprising:
administering to a subject in need thereof a therapeutically effective amount of the CD20 binding agent of claim 1.

17. The method according to claim 16, wherein the disease or disorder is cancer.

18. A method for treating a disease or disorder involving cells expressing CD20, the method comprising:
selecting a subject on the basis of detection of CD20 on the cells; and
administering to the subject a therapeutic dose of the CD20 binding agent of claim 1.

19. The method according to claim 18, wherein the disease or disorder is cancer.

20. A kit comprising the CD20 binding agent of claim 1.
